(19) United States
(12) Patent Application Publication
(10) Pub. No.: US 2002/0127205 A1
(43) Pub. Date: Sep. 12, 2002

(54) CELLS EXPRESSING IMMUNOREGULATORY MOLECULES AND USES THEREFOR

(76) Inventor: ALBERT EDGE, CAMBRIDGE, MA (US)

Correspondence Address: LAHIVE & COCKFIELD, 28 STATE STREET, BOSTON, MA, 02109, US

(21) Appl. No.: 09/144,006

(22) Filed: Aug. 31, 1998

(51) Int. Cl. A61K 48/00; C12N 15/00

(52) U.S. Cl. 424/93.2; 424/93.21; 435/325; 435/320.1

(57) ABSTRACT

Compositions comprising genetically modified cells which express at least one immunoregulatory molecule and methods for using the genetically modified cells are described. The immunoregulatory molecule expressed by the cell(s) are capable of inhibiting T cell activation and/or natural killer cell-mediated immune response against the cell upon transplantation into a recipient subject. The cells of the invention can express an immunoregulatory molecule on the surface of the cells or secrete the immunoregulatory molecule in soluble form. The cells of the invention can be transplanted into a recipient subject such that immune rejection of the cell is inhibited. In addition, non-human transgenic animals which contain cells which are genetically modified to express at least one immunoregulatory molecule are described.

### BACKGROUND OF THE INVENTION

The ability to transplant cells, tissues and organs from animals into humans as replacements for diseased human cells, tissues or organs would overcome a key limitation in clinical transplantation: the shortage of suitable human donor organs. However, the problem of immune-mediated rejection continues to hamper the clinical application of xenogeneic transplantation. Xenogeneic tissues, similar to tissues from mismatched human donors, are subject to rejection by the human cellular immune system.

The induction of an immune response to allogeneic and xenogeneic grafts requires several complex interactions between T lymphocytes and various antigen presenting cells (APC) that result in the expansion of antigen-specific cells, including B cells and T cells, the interaction of several different molecules on the surface of T cells and other cells. including accessory, adhesion and costimulatory molecules with their ligands, and ultimately, the secretion of cytokines that generally govern the outcome of the immune reaction. The initial activation and expansion of T cells is a critical step in the generation of a successful immune response against allografts and xenografts.

One approach to inhibiting T cell-mediated immune response to allogeneic and xenogeneic cells has been to treat the recipient with immunosuppressive drugs or inhibitors of complement prior to transplantation (see Bach, F. H. (1993) Transpl. Proc. 25:25-29; and Platt, J. L. and Bach, F. H. (1991) Transplantation 52:937-947). This approach has successfully prolonged the survival of xenografts for several months but suffers from the problems generally associated with administration of high doses of immunosuppressants.

A second approach to inhibiting T cell activity against an allograft or xenograft has been to administer to the transplant recipient T cell specific antibodies which deplete or sequester T cells in the recipient (see Wood et al. (1992) Neuroscience 49:410; and DeSilvia, D. R. (1991) J. Immunol. 147:3261-3267). Although enhanced graft survival has been demonstrated with T cell specific antibodies, concerns over the effectiveness of administering antibodies in vivo for human therapies has lead to the search for other methods of inhibiting xenograft and allograft rejection.

Xenotransplantation offers the benefit of an increased number of organs for transplantation. Additional methods of inhibiting transplantation rejection are needed, however, in order to take advantage of these potential organ sources.

### SUMMARY OF THE INVENTION

The present invention is based, at least in part, on the discovery that expression of immunoregulatory molecules, e.g., expression on the surface of a cell or secretion from a cell in soluble form, can provide transplanted cells with immune privilege. By decreasing T cell recognition and/or decreasing natural killer (NK) cell-mediated response to a transplanted cell, prolonged graft survival can be obtained.

In one aspect, the invention pertains to transplantable compositions comprising a cell which is genetically modified to express a first immunoregulatory molecule which inhibits T cell activation and a second immunoregulatory molecule comprising a killer inhibitor sequence, such that following transplantation of the cell into a human subject, rejection of the cell is inhibited.

In one embodiment, the first and second immunoregulatory molecules are expressed as a single soluble fusion protein. In another embodiment, the first or second immunoregulatory molecule is expressed on the surface of the cell. In yet another embodiment, the first immunoregulatory molecule is secreted by the cell.

In another embodiment, the cell is genetically modified by transfection of one or more heterologous nucleic acid molecules encoding the first and second immunoregulatory molecules such that the first and second molecules are expressed by the cell.

In a preferred embodiment, the first immunoregulatory molecule is FasL. In another preferred embodiment, the first immunoregulatory molecule is CD8. In yet another preferred embodiment, the first immunoregulatory molecule is a soluble cytokine receptor. In still another preferred embodiment, the first immunoregulatory molecule is a soluble costimulatory molecule. In yet a further preferred embodiment, the first immunoregulatory molecule is soluble CD40 or soluble CD40L.

In one embodiment, the second immunoregulatory molecule is selected from the group consisting of a human MHC class I molecule, a chimeric MHC class I molecule, or a viral MHC class I homolog. In a preferred embodiment, the second immunoregulatory molecule comprises an amino acid sequence selected from the group consisting of an HLA C or G molecule. In another preferred embodiment, the second immunoregulatory molecule is a chimeric, porcine MHC class I molecule comprising a portion of a human class I MHC molecule sufficient to render the chimeric class I molecule functional as a killer inhibitory receptor. In yet another preferred embodiment, the immunoregulatory molecule comprises an amino acid sequence selected from the group consisting of an HLA C Ser77-Asn80; HLA C Asn77-Lys80; HLA B Asn77-Arg83; and HLA A Asp74.

In one embodiment, the first or second immunoregulatory molecule is under the control of a tissue specific promoter.

In a preferred embodiment, the cell is a porcine cell. In another preferred embodiment, the cell is a fetal cell. In yet another embodiment the cell is a stem cell. In another embodiment, the cell is an embryonic stem cell. In yet another embodiment, the cell is a progenitor cell.

In another preferred embodiment, the cell is obtained from a pig which is predetermined to be free from at least one organism selected from the group consisting of zoonotic and cross-placental organisms.

In preferred embodiments, the cell is selected from the group consisting of: a pancreatic islet cell, a kidney cell, a cardiac cell, a muscle cell, a liver cell, a lung cell, an endothelial cell, a central nervous system cell, a peripheral nervous system cell, an epithelial cell, an eye cell, a skin cell, an ear cell, and a hair follicle cell.

In another aspect, the invention pertains to transplantable compositions comprising a cell which is genetically modified to express a chimeric MHC class I molecule or a viral MHC class I homolog, such that following transplantation of the xenogeneic cell into a human subject, rejection of the xenogeneic cell is inhibited.

In another preferred embodiment, the immunoregulatory molecule is a chimeric, porcine MHC class I molecule comprising a portion of a human class I MHC molecule sufficient to render the chimeric class I molecule functional as a killer inhibitory receptor. In a more preferred embodiment, the immunoregulatory molecule comprises an amino acid sequence selected from the group consisting of an HLA C Ser77-Asn80; HLA C Asn77-Lys80; HLA B Asn77-Arg83; and HLA A Asp74.

In one embodiment, the expression of the immunoregulatory molecule is under the control of a tissue specific promoter.

In a preferred embodiment, the cell is a porcine cell. In another preferred embodiment, the cell is a fetal cell. In yet another embodiment the cell is a stem cell. In another embodiment, the cell is an embryonic stem cell. In yet another embodiment, the cell is a progenitor cell.

In preferred embodiments, the cell is obtained from a pig which is predetermined to be free from at least one organism selected from the group consisting of zoonotic and cross-placental organisms.

In preferred embodiments, the cell is selected from the group consisting of: a pancreatic islet cell, a kidney cell, a cardiac cell, a muscle cell, a liver cell, a lung cell, an endothelial cell, a central nervous system cell, a peripheral nervous system cell, an epithelial cell, an eye cell, a skin cell, an ear cell, and a hair follicle cell.

In one embodiment, the compositions of the instant invention further comprise a pharmaceutically acceptable carrier.

In another aspect, the invention pertains to a method for inhibiting immune rejection of a cell comprising administering a cell which has been genetically modified to express a first immunoregulatory molecule which inhibits T cell activation and a second immunoregulatory molecule which comprises a killer inhibitor sequence, such that following transplantation of the cell into a human subject, immune rejection of the cell is inhibited.

In one embodiment, the first and second immunoregulatory molecules are expressed as a single soluble fusion protein.

In another embodiment, the first or second immunoregulatory molecule is expressed on the surface of the cell. In yet another embodiment, the first immunoregulatory molecule is secreted by the cell.

In one embodiment, the cell is genetically modified by transfection of one or more heterologous nucleic acid molecules encoding the first and second immunoregulatory molecules such that the first and second molecules are expressed by the cell.

In a preferred embodiment, the first immunoregulatory molecule is FasL. In another preferred embodiment, the first immunoregulatory molecule is CD8. In yet another preferred embodiment, the first immunoregulatory molecule is a soluble cytokine receptor. In still another preferred embodiment. the first immunoregulatory molecule is a soluble costimulatory molecule. In yet another preferred embodiment, the first immunoregulatory molecule is soluble CD40 or soluble CD40L.

In one embodiment, the second immunoregulatory molecule is selected from the group consisting of a human MHC class I molecule, a chimeric MHC class I molecule, or a viral MHC class I homolog. In preferred embodiments, the immunoregulatory molecule comprises an amino acid sequence selected from the group consisting of an HLA C or G molecule. In another preferred embodiment, the second immunoregulatory molecule is a chimeric, porcine MHC class I molecule comprising a portion of a human class I MHC molecule sufficient to render the chimeric class I molecule functional as a killer inhibitory receptor. In more preferred embodiments, the immunoregulatory molecule comprises an amino acid sequence selected from the group consisting of an HLA C Ser77-Asn80; HLA C Asn77-Lys80; HLA B Asn77-Arg83; and HLA A Asp74.

In one embodiment, the expression of the first or second immunoregulatory molecule is under the control of a tissue specific promoter.

In a preferred embodiment, the cell is a porcine cell. In another preferred embodiment, the cell is a fetal cell. In yet another embodiment the cell is a stem cell. In another embodiment, the cell is an embryonic stem cell. In yet another embodiment, the cell is a progenitor cell.

In one embodiment, the cell is obtained from a pig which is predetermined to be free from at least one organism selected from the group consisting of zoonotic and crossplacental organisms.

In preferred embodiments, the cell is selected from the group consisting of: a ancreatic islet cell, a kidney cell, a cardiac cell, a muscle cell, a liver cell, a lung cell, an ndothelial cell, a central nervous system cell, a peripheral nervous system cell, an pithelial cell, an eye cell, a skin cell, an ear cell, and a hair follicle cell.

In yet another aspect, the invention pertains to a method for inhibiting immune ejection of a cell comprising administering a xenogeneic cell which has been genetically modified to express a chimeric MHC class I molecule or a viral MHC class I homolog, such that following transplantation of the xenogeneic cell into a human subject, immune rejection of the cell is inhibited.

In another preferred embodiment, the chimeric MHC molecule is a chimeric, porcine MHC class I molecule comprising a portion of a human class I MHC molecule sufficient to render the chimeric class I molecule functional as a killer inhibitory receptor. In a more preferred embodiment, the chimeric MHC molecule comprises an amino acid sequence selected from the group consisting of an HLA C Ser77-Asn80; HLA C Asn77-Lys80; HLA B Asn77-Arg83; and HLA A Asp74.

In a further embodiment, the chimeric MHC is under the control of a tissue specific promoter.

In a preferred embodiment, the cell is a porcine cell. In another preferred embodiment, the cell is a fetal cell. In yet another embodiment the cell is a stem cell. In another embodiment, the cell is an embryonic stem cell. In yet another embodiment, the cell is a progenitor cell.

In one embodiment, the cell is obtained from a pig which is predetermined to be free from at least one organism selected from the group consisting of zoonotic and cross-placental organisms.

In preferred embodiments, the cell is selected from the group consisting of: a pancreatic islet cell, a kidney cell, a cardiac cell, a muscle cell, a liver cell, a lung cell, an endothelial cell, a central nervous system cell, a peripheral nervous system cell, an epithelial cell, an eye cell, a skin cell, an ear cell, and a hair follicle cell.

In one embodiment, the instant methods further comprise the step of administering to the subject an immunoregulatory molecule which is capable of inhibiting T cell or natural killer cell mediated immune rejection of the cell.

In yet another aspect the invention pertains to non-human transgenic animals comprising a cell which is genetically modified to express a chimeric MHC class I molecule or a viral MHC class I homolog, such that following transplantation of the cell into a human subject, immune rejection of the cell is inhibited.

In a further aspect the invention pertains to non-human transgenic animals comprising a cell which is genetically modified to express a first immunoregulatory molecule which inhibits T cell activation and a second immunoregulatory molecule which is a killer inhibitory sequence, such that following transplantation of the cell into a human subject, immune rejection of the cell is inhibited.

In preferred embodiments the non-human transgenic animal is a pig. In other preferred embodiments, the non-human transgenic animal is free from at least one organism selected from the group consisting of zoonotic and cross-placental organisms.

In another aspect, the invention pertains to a transplantable composition comprising a xenogeneic cell which is genetically modified to express an immunoregulatory molecule which inhibits T cell activation selected from the group consisting of CD8, soluble cytokine receptor, soluble costimulatory molecule, soluble CD40 and soluble CD40L, such that following transplantation of the xenogeneic cell into a human subject, rejection of the xenogeneic cell is inhibited.

In yet another aspect the invention pertains to a method for inhibiting immune rejection of a cell comprising administering a cell which has been genetically modified to express an immunoregulatory molecule which inhibits T cell activation selected from the group consisting of CD8, soluble cytokine receptor, soluble costimulatory molecule, soluble CD40 and soluble CD40L, such that following transplantation of the cell into a human subject, rejection of the cell is inhibited.

In a further aspect the invention pertains to a transplantable composition comprising a cell which is genetically modified to express an immunoregulatory molecule which inhibits T cell activation selected from the group consisting of: CD8, soluble cytokine receptor, soluble costimulatory molecule, soluble CD40 and soluble CD40L and/or a molecule comprising a killer inhibitory sequence selected from the group consisting of: a human MHC class I molecule, a chimeric MHC class I molecule, or a viral MHC class I homolog, such that following transplantation of the cell into a human subject, rejection of the cell is inhibited.

In yet another aspect, the invention pertains to a method for inhibiting immune rejection of a cell comprising administering a cell which is genetically modified to express an immunoregulatory molecule which inhibits T cell activation selected from the group consisting of: CD8, soluble cytokine receptor, soluble costimulatory molecule, soluble CD40 and soluble CD40L and/or a molecule comprising a killer inhibitory sequence selected from the group consisting of: a human MHC class I molecule, a chimeric MHC class I molecule, or a viral MHC class I homolog, such that following transplantation of the cell into a human subject, rejection of the cell is inhibited.

### DETAILED DESCRIPTION OF THE INVENTION

The present invention features cells which have been genetically modified to express an immunoregulatory molecule capable of inhibiting T cell activation and/or NK cell activation such that upon transplantation into a recipient subject, rejection of the cell is inhibited. The invention is further described in the following subsections:

Cells

Cells of the invention include cells which can be isolated or obtained in a form that can be transplanted to a subject, e.g., a xenogeneic or allogeneic subject. In a preferred embodiment, the cells are mammalian cells, e.g., human or non-human (e.g., porcine, monkey, sheep, dog, cow, goat, chicken, etc.) cells. In a particularly preferred embodiment, the mammalian cells are porcine cells. Mammalian cells, e.g., porcine cells or human cells can be adult or fetal cells. In one embodiment, the cells are stem cells. In another embodiment, the cells are embryonic stem cells. In yet another embodiment the cells are progenitor cells (e.g., pluripotential cells or multipotential cells). The cells can be in a heterogenous or homogenous cell suspension. In addition, the cells of the invention can be within a tissue or organ. Exemplary cell types for use in the invention include endothelial cells, hepatocytes, pancreatic islet cells (including α, β, δ and φ cells), muscle cells (including skeletal and cardiac myocytes and myoblasts), fibroblasts, epithelial cells, neural cells (e.g., striatal, mesencephalic and cortical cells), bone marrow cells, hematopoietic cells, eye cells (e.g., retinal pigment epithelium (RPE) cells, neural retina cells, and corneal cells), skin cells, ear cells, peripheral nerve cells, central nervous system cells, and hair follicle cells.

In another embodiment, the cells of the invention are cells which are free from at least one organism which originates in the animal from which the cells are obtained and which transmits infection or disease to a recipient subject. Cells with these characteristics can be obtained by screening the animal to determine if it is essentially free from organisms or substances which are capable of transmitting infection or disease to a recipient, e.g., a human recipient, of the cells. Typically, the cells are porcine cells which are obtained from a swine which is essentially free from pathogens which detrimentally affect humans. For example, the pathogens from which the swine are free include, but are not limited to, one or more of pathogens from the following categories of pathogens: zoonotic, cross-placental, neurotropic, hepatotropic and cardiotropic organisms. As used herein, “zoonotic” refers to organisms which can be transferred from pigs to man under natural conditions; “cross-placental” refers to organisms capable of crossing the placenta from mother to fetus; “neurotropic” refers to organisms which selectively infect neural cells; “hepatotropic” refers to organisms which selectively infect liver cells; and “cardiotropic” refers to organisms which selectively infect cardiomyoblasts or cardiomyocytes. Within each of these categories, the organism can be a parasite, bacterium, mycoplasma, or virus. For example, the swine can be free from parasites such as zoonotic parasites (e.g., toxoplasma), cross-placental parasites (e.g., eperythozoon suis or toxoplasma), neurotropic parasites (e.g., toxoplasma), hepatotropic parasites (e.g., ascarids, echinococcus, eperythozoon parvum, eperythozoon suis or toxoplasma) and/or mycoplasma, such as M. hypopneumonia. Additionally, the swine can be free from bacteria such as zoonotic bacteria (e.g., brucella, listeria, mycobacterium TB, leptospirillum), cross-placental bacteria (e.g., brucella, listeria, leptospirillum), neurotropic bacteria (e.g., listeria) and/or hepatotropic bacteria (e.g., brucella, clostridium, hemophilus suis, leptospirillum, listeria, mycobacterium TB, salmonella). Specific examples of bacteria from which the swine can be free include brucella, clostridium, hemophilus suis, listeria, mycobacterium TB, leptospirillum, salmonella and hemophilus suis. Additionally, the swine can be free from viruses such as zoonotic viruses, viruses that can cross the placenta in pregnant sows, neurotropic viruses, hepatotropic viruses and cardiotropic viruses. Zoonotic viruses include, for example, a virus in the rabies virus group, a herpes-like virus which causes pseudorabies, encephalomyocarditis virus, swine influenza Type A, transmissible gastroenteritus virus, parainfluenza virus 3 and vesicular stomatitis virus. Cross-placental viruses include, for example, viruses that cause porcine respiratory reproductive syndrome, a virus in the rabies virus group, a herpes-like virus which causes pseudorabies, parvovirus, a virus that causes swine vesicular disease, teschen (porcine polio virus), hemmaglutinating encephalomyocarditis, cytomegalovirus, suipoxvirus, and swine influenza type A. Neurotropic viruses include, for example, a virus in the rabies virus group, a herpes-like virus which causes pseudorabies, parvovirus, encephalomyocarditis virus, a virus which causes swine vesicular disease, porcine poliovirus (teschen), a virus which causes hemmaglutinating encephalomyocarditis, adenovirus, parainfluenza 3 virus. Hepatotropic viruses include, for example, a virus in the rabies virus group, bovine viral diarrhea, a herpes-like virus which causes pseudorabies, parvovirus, encephalomyocarditis virus, a virus which causes swine vesicular disease, porcine poliovirus (teschen), a virus which causes hemmaglutinating encephalomyocarditis, adenovirus, swine influenza type A virus, transmissible gastroenteritis virus, and a virus which causes (or results in) porcine respiratory reproductive syndrome. Specific examples of viruses from which the swine are free include: a virus which causes (or results in) porcine respiratory reproductive syndrome, a virus in the rabies virus group, a herpes-like virus which causes pseudorabies, parvovirus, encephalomyocarditis virus, a virus which causes swine vesicular disease, porcine poliovirus (teschen), a virus which causes hemmaglutinating encephalomyocarditis, cytomegalovirus, suipoxvirus, swine influenza type A, adenovirus, transmissible gastroenteritus virus, a virus which causes bovine viral diarrhea, parainfluenza virus 3, and vesicular stomatitis virus.

In one embodiment, the pigs from which the cells are isolated are essentially free from the following organisms: Toxoplasma, eperythrozoon, brucella, listeria, mycobacterium TB, leptospirillum, hemophilus suis, M. Hypopneumonia, a virus which causes porcine respiratory reproductive syndrome, a virus which causes rabies, a virus which causes pseudorabies, parvovirus, encephalomyocarditis virus, a virus which causes swine vesicular disease, porcine polio virus (teschen), a virus which causes hemagglutinating encephalomyocarditis, suipoxvirus, swine influenza type A, adenovirus, transmissible gastroenteritis virus, a virus which causes bovine viral diarrhea, and vesicular stomatitis virus. The phrase “essentially free from organisms or substances which are capable of transmitting infection or disease to a xenogeneic recipient” (also referred to herein as “essentially pathogen-free”) when referring to a swine from which cells are isolated or to the cells themselves means that swine does not contain organisms or substances in an amount which transmits infection or disease to a xenogeneic recipient, e.g. a human. Example VIII provides representative, but not limiting, examples of methods for selecting swine which are essentially free from various pathogens. The cells of the invention can be isolated from embryonic or post-natal swine which are determined to be essentially free of such organisms. These swine are maintained under suitable conditions until used as a source of cells for transplantation.

Immunoregulatory Molecules

The language “immunoregulatory molecule” includes those molecules which inhibit T cell and/or NK cell activity. An immunoregulatory molecule capable of inhibiting T cell activation includes molecules capable of decreasing or inhibiting T cell activity, e.g., T cell activity against the cell expressing an immunoregulatory molecule upon transplantation of the cell (e.g., donor cell) into a recipient subject, e.g., an allogeneic or xenogeneic subject. T cells play a central role in the induction of an immune response against allogeneic and xenogeneic cells. Upon introduction of an allogeneic or xenogeneic cell into a recipient subject, T cells are capable of recognizing and interacting with antigens present on the surface of the donor cell or processed antigens displayed on the surface of the recipient antigen presenting cells. The interaction of T cell receptors with antigens on the donor cell activates T cells to produce and secrete cytokines which results in the production of antigen specific cells (e.g., B cells and cytotoxic T cells) and ultimately immune rejection of the donor cell. T cells include both T helper (e.g., Th1 and Th2) cells and T killer cells. NK cells have also been found to play a role in allogeneic and xenogeneic graft rejection.

Using art recognized techniques, such as those described in further detail below, immunoregulatory molecules can be expressed by a cell of the invention. Immunoregulatory molecules can be expressed on the cell surface or can be secreted. Proteins which are normally expressed on the cell surface can be expressed in soluble form using a number of methods known in the art. For example, a nucleic acid molecule encoding a portion of the molecule which functions in immunoregulation of T and/or NK cells (e.g., an extracellular domain of the immunoregulatory molecule) can be fused to a second polypeptide sequence (e.g., an immunoglobulin sequence). The techniques for expression such soluble fusion proteins, e.g., synthesis of oligonucleotides, PCR, transforming cells, constructing vectors, expression systems, and the like are well known in the art. See for example, the contents of U.S. Pat. No. 5,580,756, the contents of which are incorporated herein by reference.

An immunoregulatory molecule expressed by a cell of the present invention can decrease the activity of an immune cell (e.g. a T or NK cell) for example by direct interaction (e.g., by delivering a veto signal to a T cell) by interacting with a factor involved in T or NK cell activation, or by competitively inhibiting T or NK cell activation. When a cell is genetically modified to express such a regulatory molecule, and transplanted into a recipient subject, cell survival is prolonged or rejection of the cell is prevented.

For example, the immunoregulatory molecule can block antigen presentation to T cells or the binding of molecules which are involved with T cell activation. In addition, the immunoregulatory molecule can bind with an antigen on the T cell surface and deliver a veto signal to T cells. For example, a donor cell expressing CD8 can act as a veto cell. The expression of CD8 on the donor cell allows delivery of a veto signal to T cells that recognize self epitopes, e.g., MHC class 1, on the donor cells thereby inactivating T cells prior to interaction with foreign antigens on the donor cell and reducing or eliminating the availability of T cells for subsequent rejection of the donor cell.

In one embodiment, the immunoregulatory molecule depletes or eliminates activated T cells in the recipient. Methods by which the immunoregulatory molecules deplete or eliminate activated T cells include T cell apoptosis and T cell inactivation. For example, activated T cells demonstrate increased expression of the glycoprotein, Fas, on their surface as compared to resting T cells. By administering donor cells which express FasL immunoregulatory molecule, the interaction (e.g., binding) of FasL to Fas induces apoptosis of activated T cells, thereby decreasing T cell activity against the cell.

Alternatively, the immunoregulatory molecule expressed by a donor cell can decrease T cell activity against the cell by preventing or reducing T cell activation.

Preferably, the immunoregulatory molecule capable of inhibiting T cell activation is selected from the group consisting of FasL, CD40L, CD40, CTLA4, CD8 and cytokine receptors. Preferably, CD40L, CD40, CTLA4, and/or cytokine receptors are expressed in soluble form (e.g., as an Ig fusion protein) by the cells of the invention. Examples of cytokine receptors include interferon gamma, TNF-α:, IL-2, IL-4, IL-6, IL-10 and IL-12 receptors. The nucleotide sequences which encode these immunoregulatory molecules are known in the art. For example, the nucleotide sequence of the cDNA encoding membrane associated human FasL is disclosed in Takahashi et al. (1994) Int. Immunol. 6(10):1567-1574, and the cDNA encoding soluble FasL is disclosed in Takahashi et al. (1994) Cell 76:969-976. In addition, the following articles describe other nucleotide sequences which encode immunoregulatory molecules, for example, CD40L (Gauchat et al. (1993) FEBS 315(3):259-266; Grafet al. (1992) Eur. J. Immunol. 22:3191-3194; Seyama (1996) Hum. Genet. 97:180-185); CD40 (Stamenlovic et al. (1988) EMBO J. 7:1053-1059); CTLA4Ig (WO 95/34320 and WO 95/33770); CD8 (Shuie et al. (1988) J. Exp. Med. 168:1993-2005; Nakayama (1989) ImmunoGenetics 30:393-397); interferon gamma receptor (Taya et al. (1982) EMBO J. 1:953-958; Gray et al. (1982) Nature 298:859-863); IL-2 receptor (Takeshita et al. (1992) Science 257:379-382; Cosman et al. (1984) Nature 312:768-771; Nikaido et al. (1984) Nature 311:626-631); IL-4 receptor (Harada et al. (1990) Proc. Natl. Acad. Sci. USA 87:857-861; Galizzi et al. (1990) Int. Immunol. 2:669-679) IL-6 receptor (Wong et al. (1988) Behringer Inst. Mitt. 83:40-47); IL-10 (Genbank™ Accession Number U16720); and IL-12 receptor (Chua et al. (1994) J. Immunol. 153:128-136). In one embodiment, a cell of the invention is genetically modified to express in immunoregulatory molecule which is not FasL

In another embodiment, the cells of the invention are modified to express a molecule which comprises a killer inhibitor sequence. A killer inhibitor sequence can inhibit NK cell-mediated or T cell mediated lysis. The language “killer inhibitor sequence” as used herein, refers to a sequence in an immunoregulatory molecule which is capable of decreasing or inhibiting T killer cell or NK cell activity against a cell expressing the immunoregulatory molecule upon transplantation of the cell into a recipient subject, e.g., an allogeneic or xenogeneic subject. For example, lysis of a donor cell by NK cells can be inhibited when an inhibitory receptor on the NK cell is engaged by a molecule on the donor cell which delivers a negative signal to the NK cell. The negative signal prevents the NK cell from lysing the donor cell, thereby allowing prolonged graft survival of the cell after transplantation into a recipient subject, e.g., a xenogeneic or allogeneic subject (Sullivan et al. (1997) J. Immunol. 159(5):2318-2326). Preferred killer inhibitor sequences include NK inhibitory sequences. A killer inhibitory sequence can be derived e.g., from human MHC class I molecule sequences (see, e.g., WO 97/06241) or viral homologs of human MHC class I sequences, e.g., cytomegalovirus sequences homologous to MHC class I. Nucleotide sequences encoding NK inhibitory sequences are known in the art. For example, the nucleotide sequence encoding human MHC class I molecule is described in Parham et al. (1988) Proc. Natl. Acad. Sci. 85:4005-4009 and the nucleotide sequence encoding cytomegalovirus MHC class I homolog is described in Beck and Barrell (1988) Nature 331:269-272.

In another embodiment, chimeric MHC class I molecules comprising killer inhibitory sequences can be expressed. As used herein the term “chimeric MHC molecule” refers to an MHC molecule composed of at least two discrete polypeptides: a first polypeptide from a human MHC molecule or viral MHC molecule homolog and a second polypeptide from porcine MHC molecule. Each of the first and second polypeptides are encoded by a nucleic acid construct and are operatively linked such that upon expression of the construct, a functional chimeric MHC molecule is produced, i.e., a fusion protein comprising the first polypeptide linked to the second polypeptide. In one embodiment, chimeric MHC class I molecules are porcine MHC class I molecules comprising a portion of a human class I MHC molecule sufficient to render the chimeric class I molecule functional as a killer inhibitory receptor. Such chimeric MHC molecules can also be constructed by making amino acid substitutions in porcine MHC class I genes using standard techniques known in the art. The portion of the chimeric MHC molecule which is human is sufficient to inhibit T killer or NK cell activity. Preferred sequences for inclusion in the chimeric MHC class I molecules of the invention can be determined, e.g., using the methods described in Example 1. For example, the amino acid sequences HLA C Ser77-Asn80; HLA C Asn77-Lys80; HLA B Asn77-Arg83; and HLA A Asp74 have been found to be sufficient to inhibit NK cell activity (Sullivan et al. (1997) J. Immunol. 159(5):2318-2326).

In another embodiment, the cell can be genetically modified to express a fusion protein. As used herein, a “fusion protein” comprises two selected polypeptides which are operatively linked to one another. For example, the fusion protein can comprise a first polypeptide which comprises an immunoregulatory molecule or a biologically active portion thereof which is capable of inhibiting T cell activation operatively linked to a second polypeptide which is capable of inhibiting T killer cells or NK cells. Preferably, the fusion protein comprises an immunoregulatory molecule or biologically active portion thereof operatively linked to a polypeptide which comprises a killer inhibitory sequence. With reference to the fusion protein, the term “operatively linked” is intended to mean that the polypeptide comprising the immunoregulatory molecule capable of inhibiting T cell activation and the killer inhibitory sequence are fused inframe frame to each other. The polypeptide containing amino acid residues critical for the inhibition of T killer or NK cell-mediated rejection (the killer inhibitory sequence) can be fused to the N-terminus or the C-terminus of the immunoregulatory molecule capable of inhibiting T cell activation in a recipient subject. In one embodiment, the fusion protein which is expressed by the cells is a soluble fusion protein. In another embodiment, the fusion protein is expressed on the surface of the cell.

Preferably, the nucleic acid molecules encoding the fusion proteins of the invention are produced by standard DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in molecular Biology, eds. Ausubel et al. John Wiley & Sons: 1992).

A “biologically active portion” of an immunoregulatory molecule is intended to include a portion of an immunoregulatory molecule which possesses a function of the immunoregulatory molecule. Biologically active portions of several immunoregulatory molecules are known in the art. For example, as described in Takahashi et al. (1994) Cell 76:969-976, amino acid residues 103 to 281 of FasL represent a soluble form of FasL which retains its ability to inhibit T cell activation. Moreover, standard binding assays known in the art can be performed to determine the ability of an immunoregulatory molecule or a biologically active portion thereof to interact with (e.g., bind to) a T cell or a factor associated with T cell-mediated immune rejection.

Moreover, it will be appreciated by those skilled in the art that nucleic acids encoding peptides having the activity of an immunoregulatory molecule but differing in sequence from a naturally occurring immunoregulatory molecule can be identified as described herein and used to genetically modify cells. For example, the DNA sequence of a known immunoregulatory molecule can be modified by genetic techniques to produce proteins or peptides with altered amino acid sequences, buth which retain their function. Such sequences are considered within the scope of the present invention, where the expressed peptide is capable of either inhibiting a T cell mediated or NK cell mediated immune response.

For example, mutations can be introduced into a DNA encoding naturally occurring immunoregulatory molecules (e.g., molecules which inhibit T cell activation or which function as killer inhibitory molecules) by any one of a number of methods, including those for producing simple deletions or insertions, systematic deletions, insertions or substitutions of clusters of bases or substitutions of single bases, to generate variants or modified equivalents of known immunoregulatory molecules. Site directed mutagenesis systems are well known in the art. Protocols and reagents can be obtained commercially from Amersham International PLC, Amersham, U.K.

Peptides having an activity of a immunoregulatory molecule, i.e., the ability to inhibit T cell activation and/or inhibit NK cell activation, as evidenced by, for example, inhibiting cytokine production, inhibit T cell proliferation, causing T cell anergy, causing apoptosis, and/ or inhibiting T cell or NK cell lysis of target cells.

Screening the peptides for those which have the characteristic of an immunoregulatory molecule can be accomplished using one or more of several different assays. For example, the peptides can be screened for by transfecting a cell, (e.g., an allogeneic or xenogeneic cell) with a nucleic acid molecule encoding a putative immunoregulatory molecule. The ability of the transfected cell to induce a T cell or an NK cell response can then be tested in a standard in vitro assay (e.g., measuring proliferation, cytokine production, anergy, or killing) or in an in vivo assay which measures the immune response of a recipient to a transplant by determining whether the transplant is rejected (e.g., either histologically or functionally) using techniques which are well known in the art. Comparisons can then be made between the untransfected allogeneic or xenogeneic cell and the cell bearing the putative immunoregulatory molecule. A functional immunoregulatory molecule can be easily identified by inducing lower T cell or NK cell responses when compared to the untransfected control cell.

In addition to the immunoregulatory molecules described above, other immunoregulatory molecules which can be used to genetically modify cells can be readily identified using techniques which are well known in the art. For example, as described above, the ability of the transfected cell to induce a T cell or an NK cell response can then be tested in a standard in vitro assay. Comparisons can then be made between the untransfected allogeneic or xenogeneic cell and the cell bearing the putative immunoregulatory molecule. A functional immunoregulatory molecule can be easily identified by diminishing T cell or NK cell responses when compared to the untransfected control cell.

To determine whether, for example, the mechanism of rejection that is inhibited is NK cell-mediated rejection, NK cells can be isolated from the recipient subject's circulation or from a site in or near the graft (e.g., from a lymph node draining the graft area), or from a tissue section of the graft. The NK cells can then be cultured and their response to cells of the same type as those that were transplanted into the recipient subject can be measured. If the NK cells appear nonresponsive to the transplant cells relative to control NK cells or NK cells cultured under the same conditions, then NK cell activity is inhibited. To determine whether, for example, the mechanism of rejection that is inhibited is T cell-mediated rejection, the above experiments can be repeated wherein T cells are substituted for NK cells.

Modification of Class I Molecules

In one embodiment, the cells of the invention can be further modified such that they possess characteristics which render them further suitable for transplantation, i.e., such that rejection of the cell is reduced by altering the cell prior to transplantation into an allogeneic or xenogeneic recipient. For example, an antigen on the surface of the cell can be altered such that an immune response against the cell is reduced as compared to unaltered cells. In an unaltered state, the antigen on the cell surface stimulates an immune response against the cell when the cell is administered to a recipient subject. By altering the antigen, the normal immunological recognition of the donor cell by the immune system cells of the recipient is disrupted. In addition, this altered immunological recognition of the antigen can lead to cell-specific long term unresponsiveness in the recipient. It is likely that alteration of an antigen on the surface of a cell prior to introducing the cell into a subject interferes with the initial phase of recognition of the donor cell by the cells of the host's immune system subsequent to administration of the cell. Furthermore, alteration of the antigen can induce immunological nonresponsiveness or tolerance, thereby preventing the induction of the effector phases of an immune response (e.g., cytotoxic T cell generation, antibody production etc.) which are ultimately responsible for rejection of foreign cells in a normal immune response. As used herein, the term “altered” encompasses changes that are made to at least one cell surface antigen which reduce the immunogenicity of the antigen to thereby interfere with immunological recognition of the antigen(s) by the recipient's immune system. An example of an alteration of a cell surface antigen is binding of a second molecule to the antigen. The second molecule can decrease or prevent recognition of the antigen as a foreign antigen by the recipient subject's immune system.

The antigen on the mammalian cell to be altered can be an MHC class I antigen. Alternatively, an adhesion molecule on the cell surface, such as NCAM-1 or ICAM-1, can be altered. An antigen which stimulates a cellular immune response against the cell, such as an MHC class I antigen, can be altered prior to transplantation by contacting the cell with a molecule which binds to the antigen. A preferred molecule for binding to the antigen is an antibody, or fragment thereof (e.g., an anti-MHC class I antibody, or fragment thereof, an anti-ICAM-1 antibody or fragment thereof, an anti-LFA-3 antibody or fragment thereof, or an anti-β₂ microglobulin antibody or fragment thereof). A preferred antibody fragment is an F(ab′)₂ fragment. Polyclonal or, more preferably, monoclonal antibodies can be used. Other molecules which can be used to alter an antigen (e.g., an MHC class I antigen) include peptides and small organic molecules which bind to the antigen. Furthermore, two or more different epitopes on the same or different antigens on the cell surface can be altered. A particularly preferred monoclonal antibody for alteration of MHC class I antigens on porcine cells is PT85 (e.g., PT85A or PT85B; commercially available from Veterinary Medicine Research Development, Pullman, Wash.). PT85 can be used alone to alter MHC class I antigens or, if each antibody is specific for a different epitope, PT85 can be used in combination with another antibody known to bind MHC class I antigens to alter the antigens on the cell surface. The antibody W6/32 can also be used. Suitable methods for altering a surface antigen on a cell for transplantation are described in greater detail in Faustman and Coe (1991) Science 252:1700-1702 and PCT Publication Number WO 92/04033. Methods for altering multiple epitopes on a surface antigen on a cell for transplantation are described in greater detail in PCT Publication Number WO 95/26740 published on Oct. 12, 1995, the contents of which are incorporated herein by reference.

An epitope on the cell can also be altered, reduced or substantially eliminated in order to reduce natural antibody-mediated hyperacute rejection of the cell. Preferably, the epitope which is altered is a galactosyl(α1-3)galactose epitope. In one embodiment, expression of alpha-galactosyl epitopes on a cell surface is reduced or substantially eliminated by introducing into the cell a nucleic acid, e.g., cDNA which is antisense to a regulatory or coding region of an alpha-galactosyl-transferase gene (e.g., a pig alpha-galactosyltransferase gene in a porcine cell). Alternatively, a cell can be contacted with (e.g., incubated with) an oligonucleotide antisense to a glycosyltransferase gene, or infected with a viral vector containing nucleic acid antisense to a glycosyltransferase gene, to inhibit the activity of an alpha-galactosyltransferase in the cell. Methods for altering an antigen such that natural antibody mediated rejection is inhibited are described in greater detail in PCT Publication Number WO 95/33828 published on Dec. 14, 1995, the contents of which are incorporated herein by reference.

Genetic Modification of Cells

The cells of the invention are genetically modified to express an immunoregulatory molecule. As used herein, the language “genetically modified to express” is intended to mean that the cell is treated in a manner that results in the production of an immunoregulatory molecule by the cell. Preferably, the cell does not express the gene product prior to the modification. Alternatively, genetic modification of the cell can result in an increased production of a gene product already expressed in the cell.

In a preferred embodiment, the cell is genetically modified to express an immunoregulatory molecule by introducing genetic material, such as a nucleic acid molecule, e.g., RNA, or more preferably, DNA, into the cell. The nucleic acid introduced into the cell encodes an immunoregulatory molecule to be expressed by the cell. As used herein, the term “express” refers to the production of an observable phenotype by a gene, e.g., synthesis of a protein. The immunoregulatory molecule can be expressed on the surface of the cell or secreted from the cell in a soluble form. Furthermore, the immunoregulatory molecule can be generally expressed or can be under the control of a tissue specific promoter.

A nucleic acid molecule introduced into a cell is in a form suitable for expression in the cell of the immunoregulatory molecule encoded by the nucleic acid. Accordingly, the nucleic acid molecule includes coding and regulatory sequences required for transcription of the gene (or portion thereof) and translation of the immunoregulatory molecule encoded by the gene. Regulatory sequences which can be included in the nucleic acid molecule include promoters, enhancers, and polyadenylation signals, as well as sequences necessary for transport of an encoded protein or peptide, for example N-terminal signal sequences for transport of proteins or peptides to the Golgi apparatus and the surface of the cell for secretion.

Nucleotide sequences which regulate the expression of a gene product (e.g., promoter and enhancer sequences) can be selected based upon the type of cell in which the immunoregulatory molecule is to be expressed and the desired level of expression. In a preferred embodiment, a promoter known to confer cell-type specific expression of a gene linked to the promoter can be used. Tissue-specific regulatory elements are known in the art, for example, an albumin promoter or major urinary protein (MUP) promoter can be used for liver-specific expression; insulin regulatory elements can be used for pancreatic islet cell-specific expression; and, various neural cell-specific regulatory elements, including neuron-specific enolase, tyrosine hydroxlase and dopamine D2 receptor can be used for neurospecific expression. Alternatively, a regulatory element which can direct constitutive expression of a gene in a variety of different cell-types can be used. Promoters for general expression of immunoregulatory molecules include, for example, the β-actin promoter and the H2K^{b} promoter. In addition, viral regulatory elements can be used for general expression of immunoregulatory molecules. Examples of viral promoters commonly used to drive gene expression include those derived from polyoma virus, Adenovirus 2, cytomegalovirus and Simian Virus 40, and retroviral LTRs. Alternatively, a regulatory element which provides inducible expression of a gene linked thereto can be used. The use of an inducible regulatory element (e.g., an inducible promoter) allows for modulation of the production of the gene product in the cell. Examples of potentially useful inducible regulatory systems for use in eukaryotic cells include hormoneregulated elements (e.g., see Mader, S. and White, J. H. (1993) Proc. Natl. Acad Sci. USA 90:5603-5607), synthetic ligand-regulated elements (see, e.g. Spencer, D. M. et al. (1993) Science 262:1019-1024) and ionizing radiation-regulated elements (e.g., see Manome, Y. et al. (1993) Biochemistry 32:10607-10613; Datta, R. et al. (1992) Proc. Natl. Acad. Sci. USA 89:10149-10153). Additional tissue-specific or inducible regulatory systems which may be developed can also be used in accordance with the invention.

There are a number of techniques known in the art for introducing genetic material into a cell that can be applied to modify a cell of the invention. In one embodiment, the nucleic acid is in the form of a naked nucleic acid molecule. In this embodiment, the nucleic acid molecule introduced into a cell to be modified typically includes the nucleic acid encoding an immunoregulatory molecule and the necessary regulatory elements in a plasmid. Examples of plasmid expression vectors include CDM8 (Seed, B. (1987) Nature 329:840) and pMT2PC (Kaufman, et al. (1987) EMBO J. 6:187-195). In another embodiment, the nucleic acid molecule to be introduced into a cell is contained within a viral vector. In this embodiment, the nucleic acid encoding an immunoregulatory molecule is inserted into the viral genome (or a partial viral genome). The regulatory elements directing the expression of the immunoregulatory molecule can be included with the nucleic acid inserted into the viral genome (i.e., linked to the gene inserted into the viral genome) or can be provided by the viral genome itself. Examples of methods which can be used to introduce naked nucleic acid into cells and viral-mediated transfer of nucleic acid into cells are described separately in the subsections below.

A. Introduction of Naked Nucleic Acid into Cells

Several methods are known in the art for introducing naked DNA into cells. For example, naked DNA can be introduced into cells by forming a precipitate containing the DNA and calcium phosphate. This method includes mixing a HEPES-buffered saline solution with a solution containing calcium chloride and DNA to form a precipitate. The precipitate is then incubated with cells. A glycerol or dimethyl sulfoxide shock step can be added to increase the amount of DNA taken up by certain cells. CaPO₄-mediated transfection can be used to stably (or transiently) transfect cells and is only applicable to in vitro modification of cells. Protocols for CaPO₄-mediated transfection can be found in Current Protocols in Molecular Biology, Ausubel, F. M. et al. (eds.) Greene Publishing Associates, (1989), Section 9.1 and in Molecular Cloning: A Laboratory Manual, 2nd Edition, Sambrook et al. Cold Spring Harbor Laboratory Press, (1989), Sections 16.32-16.40 or other standard laboratory manuals.

Alternatively, naked DNA can be introduced into cells by forming a mixture of the DNA and DEAE-dextran and incubating the mixture with the cells. A dimethylsulfoxide or chloroquine shock step can be added to increase the amount of DNA uptake. DEAE-dextran transfection is only applicable to in vitro modification of cells and can be used to introduce DNA transiently into cells but is not preferred for creating stably transfected cells. Thus, this method can be used for short term production of an immunoregulatory molecule but is not a method of choice for longterm production of the immunoregulatory molecule. Protocols for DEAE-dextran-mediated transfection can be found in Current Protocols in Molecular Biology, Ausubel, F. M. et al. (eds.) Greene Publishing Associates, (1989), Section 9.2 and in Molecular Cloning: A Laboratory Manual, 2nd Edition, Sambrook et al. Cold Spring Harbor Laboratory Press, (1989), Sections 16.41-16.46 or other standard laboratory manuals.

In addition, naked DNA can also be introduced into cells by incubating the cells and the DNA together in an appropriate buffer and subjecting the cells to a high-voltage electric pulse. The efficiency with which DNA is introduced into cells by electroporation is influenced by the strength of the applied field, the length of the electric pulse, the temperature, the conformation and concentration of the DNA and the ionic composition of the media. Electroporation can be used to stably (or transiently) transfect a wide variety of cell types and is only applicable to in vitro modification of cells. Protocols for electroporating cells can be found in Current Protocols in Molecular Biology, Ausubel, F. M. et al. (eds.) Greene Publishing Associates, (1989), Section 9.3 and in Molecular Cloning: A Laboratory Manual, 2nd Edition Sambrook et al. Cold Spring Harbor Laboratory Press, (1989), Sections 16.54-16.55 or other standard laboratory manuals.

Liposome-mediated transfection (“lipofection”) can also be used to introduce naked DNA into a cell. Naked DNA can be introduced into cells by mixing the DNA with a liposome suspension containing cationic lipids. The DNA/liposome complex is then incubated with cells. Liposome mediated transfection can be used to stably (or transiently) transfect cells in culture in vitro. Protocols can be found in Current Protocols in Molecular Biology, Ausubel, F. M. et al. (eds.) Greene Publishing Associates, (1989), Section 9.4 and other standard laboratory manuals. Additionally, gene delivery in vivo has been accomplished using liposomes. See for example Nicolau et al. (1987) Meth. Enz. 149:157-176; Wang and Huang (1987) Proc. Natl. Acad. Sci. USA 84:7851-7855; Brigham et al. (1989) Am. J. Med Sci. 298:278; and Gould-Fogerite et al. (1989) Gene 84:429-438.

Another method for introducing naked DNA into cells is by directly injecting the DNA into the cells. For an in vitro culture of cells, DNA can be introduced by microinjection. Since each cell is microinjected individually, this approach is very labor intensive when modifying large numbers of cells. However, a situation wherein microinjection is a method of choice is in the production of transgenic animals (discussed in greater detail below). In this situation, the DNA is stably introduced into a fertilized oocyte which is then allowed to develop into an animal. The resultant animal contains cells carrying the DNA introduced into the oocyte. Direct injection has also been used to introduce naked DNA into cells in vivo (see e.g., Acsadi et al. (1991) Nature 332: 815-818; Wolff et al. (1990) Science 247:1465-1468). A delivery apparatus (e.g., a “gene gun”) for injecting DNA into cells in vivo can be used. Such an apparatus is commercially available (e.g., from BioRad, Cambridge, Mass.).

Alternatively, naked DNA can also be introduced into cells by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. Receptors to which a DNA-ligand complex have targeted include the transferrin receptor and the asialoglycoprotein receptor. A DNA-ligand complex linked to adenovirus capsids which naturally disrupt endosomes, thereby releasing material into the cytoplasm can be used to avoid degradation of the complex by intracellular lysosomes (see for example Curiel et al. (1991) Proc. Natl. Acad. Sci. USA 88:8850; Cristiano et al. (1993) Proc. Natl. Acad. Sci. USA 90:2122-2126). Receptor-mediated DNA uptake can be used to introduce DNA into cells either in vitro or in vivo and, additionally, has the added feature that DNA can be selectively targeted to a particular cell type by use of a ligand which binds to a receptor selectively expressed on a target cell of interest.

Generally, when naked DNA is introduced into cells in culture (e.g., by one of the transfection techniques described above) only a small fraction of cells (about 1 out of 10⁵) typically integrate the transfected DNA into their genomes (i.e., the DNA is maintained in the cell episomally). Thus, in order to identify cells which have taken up exogenous DNA, it is advantageous to transfect nucleic acid encoding a selectable marker into the cell along with the nucleic acid(s) of interest. Preferred selectable markers include those which confer resistance to drugs such as neomyocin, G418, hygromycin and methotrexate. Selectable markers may be introduced on the same plasmid as the gene(s) of interest or may be introduced on a separate plasmid.

B. Viral-Mediated Gene Transfer

Another approach for introducing nucleic acid encoding an immunoregulatory molecule into a cell is by use of a viral vector containing nucleic acid, e.g. a cDNA, encoding the immunoregulatory molecule. Infection of cells with a viral vector has the advantage that a large proportion of cells receive the nucleic acid, which can obviate the need for selection of cells which have received the nucleic acid. Additionally, molecules encoded within the viral vector, e.g., by a cDNA contained in the viral vector, are expressed efficiently in cells which have taken up viral vector nucleic acid and viral vector systems can be used either in vitro or in vivo.

Defective retroviruses are well characterized for use in gene transfer for gene therapy purposes (for a review see Miller, A. D. (1990) Blood 76:271). A recombinant retrovirus can be constructed having a nucleic acid encoding an immunoregulatory molecule inserted into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψCrip, ψCre, ψ2 and ψAm. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Publication Number WO 89/07136; PCT Publication Number WO 89/02468; PCT Publication Number WO 89/05345; and PCT Publication Number WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

The genome of an adenovirus can be manipulated such that it encodes and expresses an immunoregulatory molecule but is inactivated in terms of its ability to replicate in a normal lytic viral life cycle. See for example Berkner et al. (1988) BioTechniquies 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Alternatively, adeno-associated virus (AAV) can be used to introduce a gene encoding an immunoregulatory molecule into a cell. AAV is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

The efficacy of a particular expression vector system and method of introducing nucleic acid into a cell can be assessed by standard approaches routinely used in the art. or example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The immunoregulatory molecule can be detected by an appropriate assay, for example by immunological detection of the molecule, such as with a specific antibody, or by a functional assay to detect a functional activity of the immunoregulatory molecule, such as an enzymatic assay. For example, a functional in vitro assay can include exposing cells which express an immunoregulatory molecule to human T cells in order to measure the inhibition of proliferation or induction of anergy in the T cells. If the immunoregulatory molecule to be expressed by a cell is not readily assayable, an expression system can first be optimized using a reporter gene linked to the regulatory elements and vector to be used. The reporter gene encodes a gene product which is easily detectable and, thus, can be used to evaluate the efficacy of the system. Standard reporter genes used in the art include genes encoding β-galactosidase, chloramphenicol acetyl transferase, luciferase and human growth hormone.

When the method used to introduce nucleic acid into a population of cells results in modification of a large proportion of the cells and efficient expression of the immunoregulatory molecule by the cells (e.g., as is often the case when using a viral expression vector), the modified population of cells may be used without further isolation or subcloning of individual cells within the population. That is, there may be sufficient production of an immunoregulatory molecule by the population of cells such that no further cell isolation is needed. Alternatively, it may be desirable to grow a homogenous population of identically modified cells from a single modified cell to isolate cells which efficiently express an immunoregulatory molecule. Such a population of uniform cells can be prepared by isolating a single modified cell by limiting dilution cloning followed by expanding the single cell in culture into a clonal population of cells by standard techniques.

C. Other Methods for Modifying a Cell to Express a Gene Product

Alternative to introducing a nucleic acid molecule into a cell to modify the cell to express an immunoregulatory molecule, a cell can be modified by inducing or increasing the level of expression of the immunoregulatory molecule by a cell. For example, a cell may be capable of expressing a particular immunoregulatory molecule but fails to do so without additional treatment of the cell. Similarly, the cell may express insufficient amounts of the immunoregulatory molecule to inhibit rejection of the cell upon transplantation. Thus, an agent which stimulates expression of an immunoregulatory molecule can be used to induce or increase expression of the immunoregulatory molecule by the cell. For example, cells can be contacted with an agent in vitro in a culture medium. The agent which stimulates expression of an immunoregulatory molecule may function, for instance, by increasing transcription of the gene encoding the immunoregulatory molecule, by increasing the rate of translation or stability (e.g., a post transcriptional modification such as a poly A tail) of an mRNA encoding the molecule or by increasing stability, transport or localization of the immunoregulatory molecule. Examples of agents which can be used to induce expression of an immunoregulatory molecule include cytokines and growth factors.

Another type of agent which can be used to induce or increase expression of an immunoregulatory molecule by a cell is a transcription factor which upregulates transcription of the gene encoding the molecule. A transcription factor which upregulates the expression of a gene encoding an immunoregulatory molecule can be provided to a cell, for example, by introducing into the cell a nucleic acid molecule encoding the transcription factor. Thus, this approach represents an alternative type of nucleic acid molecule which can be introduced into the cell (for example by one of the previously discussed methods). In this case, the introduced nucleic acid does not directly encode an immunoregulatory molecule but rather causes production of the immunoregulatory molecule by the cell indirectly by inducing expression of the molecule.

In yet another method, a cell is modified to express an immunoregulatory molecule by coupling the immunoregulatory molecule to the cell, preferably to the surface of the cell. For example, an immunoregulatory molecule can be obtained by purifying the cell from a biological source or expressing the protein recombinantly using standard recombinant DNA technology. The isolated protein can then be coupled to the cell. The terms “coupled” or “coupling” refer to a chemical, enzymatic or other means (e.g., by binding to an antibody on the surface of the cell or genetic engineering of linkages) by which an immunoregulatory molecule can be linked to a cell such that the immunoregulatory molecule is in a form suitable for delivering the molecule to a subject. For example, a protein can be chemically crosslinked to a cell surface using commercially available crosslinking reagents (Pierce, Rockford Ill.). Other approaches to coupling a gene product to a cell include the use of a bispecific antibody which binds both an immunoregulatory molecule and a cell-surface molecule on the cell or modification of the gene product to include a lipophilic tail (e.g., by inositol phosphate linkage) which can insert into a cell membrane.

Transyenic Animals

An alternative method for generating a cell that is modified to express an immunoregulatory molecule involves introducing naked DNA into cells to create a transgenic animal which contains cells modified to express the desired immunoregulatory molecule. Accordingly, the invention also features a non-human transgenic animal comprising a cell (or cells) which is genetically modified to express an immunoregulatory molecule which is capable of inhibiting T cell activation and/or an immunoregulatory molecule which is capable of inhibiting NK cell-mediated rejection. In a preferred embodiment, the nucleic acid molecule which encodes an immunoregulatory molecule can be introduced into a fertilized oocyte or an embryonic stem cell. Such host cells can then be used to create non-human transgenic animals in which exogenous immunoregulatory molecule sequences have been introduced into their genome. As used herein, a “transgenic animal” is a non-human animal, preferably a mammal, more preferably a pig, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded immunoregulatory molecule in one or more cell types or tissues of the transgenic animal.

A transgenic animal of the invention can be created by introducing a nucleic acid molecule encoding an immunoregulatory molecule into the male pronuclei of a fertilized oocyte, e.g., by microinjection or retroviral infection, and allowing the oocyte to develop in a pseudopregnant female foster animal. Intronic sequences and polyadenylation signals can also be included in the transgene to increase the efficiency of expression of the transgene. In addition, the gene encoding the immunoregulatory molecule can be introduced in a form engineered to direct expression of the protein on the cell surface or in a soluble form suitable for secretion. A tissue-specific regulatory sequence(s) can be operably linked to the cDNA encoding an immunoregulatory molecule to direct expression of the immunoregulatory molecule to particular cells. Methods for generating transgenic animals via embryo manipulation and microinjection, particularly animals such as mice, have become conventional in the art and are described, for example, in U.S. Pat. Nos. 4,736,866 and 4,870,009 and Hogan, B. et al., (1986) A Laboratory Manual, Cold Spring Harbor, N.Y., Cold Spring Harbor Laboratory. Similar methods are used for production of other transgenic animals, for example, methods for generating transgenic swine are described in U.S. Pat. No. 5,523,226. A transgenic founder animal can be identified based upon the presence of the transgene encoding an immunoregulatory molecule in its genome and/or expression of an immunoregulatory molecule mRNA in tissues or cells of the animals. A transgenic founder animal can then be used to breed additional animals carrying the transgene. Moreover, transgenic animals carrying a transgene encoding an immunoregulatory molecule can further be bred to other transgenic animals carrying other transgenes, e.g., other immunoregulatory molecules.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl Acad. Sci. 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT Publication Numbers WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring borne of this female foster animal will be a clone of the animal from which the cell, e.g., the somatic cell, is isolated.

In a preferred embodiment, a nucleic acid sequence encoding a human immunoregulatory molecule is introduced as a transgene into the genome of a non-human animal, e.g., a pig. For example, by methods described herein, a human cDNA encoding an immunoregulatory molecule can be introduced into the male pronuclei of a fertilized porcine oocyte. The porcine oocyte is allowed to develop in a pseudopregnant foster pig and the transgenic fetal pig can be carried to term or removed from the foster pig at a desired gestational age. Cells of the transgenic pig which contain the transgene encoding immunoregulatory molecule can then be used as a source of cells for transplantation into a human recipient. The human nucleic acid sequence to be introduced as a transgene can encode an immunoregulatory molecule capable of inhibiting T cell activation and/or an immunoregulatory molecule capable of inhibiting NK cell-mediated rejection in a human recipient. Examples of transgenes which encode immunoregulatory molecules capable of inhibiting T cell activation include human cDNA sequence encoding FasL, CD40, CD40L, CTLA4Ig, CD8 and a cytokine receptor. In addition, the transgene can be cDNA encoding an immunoregulatory molecule capable of inhibiting NK cell-mediated rejection in a human recipient, for example, a human MHC class I molecule inhibitory sequence or a cytomegalovirus protein with sequences homologous to MHC class I molecule inhibitory sequences.

In another embodiment, the transgene introduced into a porcine oocyte is a fusion protein which is capable of inhibiting NK cell-mediated rejection and T cell activation in humans. The transgene can include a porcine gene which has been modified, e.g., by site directed mutagenesis, to contain nucleic acid sequences encoding a polypeptide having amino acid residues critical for inhibiting NK cell-mediated rejection in a human recipient fused to a polypeptide which is capable of inhibiting T cell activation in humans. For example, the gene encoding a class I molecule in pig can be modified by mutagenesis to encode amino acid residues of human class I molecules shown to be critical for inhibiting NK cell-mediated rejection in humans. Amino acid residues which are critical for inhibiting NK cell-mediated rejection in humans for NK clones known in the art include, e.g., Lys⁸⁰ and possibly Asn⁷⁷ of group 1 human NK clones; Ser⁷⁷ and Asn⁸⁰ of group 2 human NK clones; or Ile⁸⁰ of group 3 human NK clones. For greater detail, see Sullivan et al. (1997) J. Immunol. 159(5):2318-2326, the contents of which are incorporated herein by reference. The polypeptide having amino acid residues critical for inhibiting NK-cell mediated rejection can be operatively linked to an immunoregulatory molecule or biologically active portion thereof which inhibits T cell activation in humans by methods known in the art and described herein.

Use of Genetically Modified Cells in Transplantation p Preferably, the cell types for use in the method of the invention are cells which can provide a therapeutic function in a disease or disorder. For example, liver cells can be transplanted into a subject with hepatic cell dysfunction (e.g., liver failure, hypercholesterolemia, hemophilia or inherited emphysema); pancreatic islet cells can be transplanted into a subject suffering from diabetes; neural cells can be transplanted into a subject suffering from Parkinson's disease, Huntington's disease, focal epilepsy or stroke, amyotrophic lateral sclerosis, pain, or multiple sclerosis; muscle cells can be transplanted into subjects suffering from a muscular dystrophy (e.g., Duchenne muscular dystrophy); cardiomyocytes or skeletal myoblasts can be transplanted into a subject displaying insufficient cardiac function (e.g., ischemic heart disease or congestive heart failure); hematopoietic cells can be transplanted into patients with hematopoietic or immunological dysfunction and neural retina or retinal pigment epithelium (RPE) cells can be transplanted into a subject with a retinal disorder (e.g., retinitis pigmentosa or macular degeneration).

Liver tissue can be obtained, for example, from brain dead human donors or from non-human animals such as pigs. The cells can be dissociated by digestion with collagenase. Viable cells can be obtained and washed by centrifugation, elution, and resuspension. The cells can be genetically modified to express at least one immunoregulatory molecule prior to isolation by obtaining the hepatocytes from a transgenic animal or after isolation of the hepatocytes, as described herein. Following genetic modification, cells are administered to the liver of the recipient patient by methods known in the art. For example, common methods of administering hepatocytes to recipient subjects, particularly human subjects, include intraperitoneal injection of the cells, (Wilson, J. et al. (1991) Clin. Biotech. 3(1):21-25), intravenous infusion of the cells into, for example, the portal vein (Kay, M. (1993) Cell Trans. 2:405-406; Tejera, J. L. et al. (1992) Transplan. Proc. 24(1):160-161; Wiederkehr, J. C. et al. (1990) Transplantation 50(3):466-476; Gunsalus et al. (1997) Nat. Med. 3:48-53; or the mesenteric vein (Grossman, M. et al. (1994) Nature Gen. 6:335-341; Wilson, J. M. et al. (1990) Proc. Natl. Acad. Sci. 87:8437-8441), intrasplenic injection of the cells (Rhim, J. A. et al. (1994) Science 263:1149-1152; Kay, M. A. (1993) Cell Trans. 2:405-406; Wiederkehr, J. C. et al. (1990) Transplantation 50(3):466-476), and infusion of the cells into the splenic artery. To facilitate transplantation of the hepatocytes into, for example, the peritoneal cavity, the cells can bound to microcarrier beads such as collagen-coated dextran beads (Pharmacia, Uppsala, Sweden) (Wilson, J. et al. (1991) Clin. Biotech. 3(1):21-25). Cells can be administered in a pharmaceutically acceptable carrier or diluent as described herein. A human liver typically consists of about 2×10¹¹ hepatocytes. To treat insufficient liver function in a human subject, about 10⁹-10¹⁰ hepatocytes are transplanted into the recipient subject.

Non-limiting examples of adverse effects or symptoms of liver disorders which the hepatocytes of the present invention can be administered to decrease or ameliorate liver dysfunction include: high serum cholesterol and early onset atherosclerosis associated with familial hypercholesterolemia; absent glucuronyl transferase activity, impaired biliary excretion, severe unconjugated hyperbilirubinemia, and neurological damage associated with Crigler-Najjar Syndrome Type I; decreased glucuronyl transferase activity and unconjugated hyperbilirubinemia associated with Gilbert's Syndrome; cirrhosis and liver failure associated with chronic hepatitis or other causes such as alcohol abuse; death in infancy associated with OTC deficiency; alveolar tissue damage associated with hereditary emphysema; deficiency in clotting factor IX associated with hemophilia B. For additional examples of adverse effects or symptoms of a wide variety of liver disorders, see Robbins, S. L. et al. Pathological Basis of Disease (W. B. Saunders Company, Philadelphia, 1984) pp. 884-942. Transplantation of hepatocytes of the invention into a subject with a liver disorder results in replacement of lost or damaged hepatocytes and replacement of liver function.

In another embodiment, pancreatic cells which have been obtained from a donor, e.g., a brain dead human donor or a non-human animal, can be isolated by enzyme digestion, centrifugation, elution and resuspension of the pancreatic islet cells. The islet cells can be genetically modified to express an immunoregulatory molecule prior to isolation by obtaining the cells from a non-human transgenic animal or the cells can be genetically modified after isolation by the methods described herein. Cells expressing an immunoregulatory molecule are then administered to a recipient subject. Common methods of administering pancreatic cells to recipient subjects, particularly human subjects, include implantation of cells in a pouch of omentum (Yoneda, K. et al. (1989) Diabetes 38 (Suppl. 1):213-216), intraperitoneal injection of the cells, (Wahoff, D. C. et al. (1994) Transplant. Proc. 26:804), implantation of the cells under the kidney capsule of the subject (See, e.g., Liu, X. et al. (1991) Diabetes 40:858-866; Korsgren, O. et al. (1988) Transplantation 45(3):509-514; Simeonovic, D. J. et al. (1982) Aust. J. Exp. Biol. Med. Sci. 60:383), and intravenous injection of the cells into, for example, the portal vein (Braesch, M. K. et al. (1992) Transplant. Proc. 24(2):679-680; Groth, C. G. et al. (1992) Transplant. Proc. 24(3):972-973). To facilitate transplantation of the pancreatic cells under the kidney capsule, the cells can be embedded in a plasma clot prepared from, e.g., plasma from the recipient subject (Simeonovic, D. J. et al. (1982) Aust. J. Exp. Biol. Med. Sci. 60:383) or a collagen matrix. Cells can be administered in a pharmaceutically acceptable carrier or diluent as described herein. To treat a human having a disease characterized by insufficient insulin activity about 10⁶-10⁷ pancreatic cells are required.

Insufficient insulin activity for which the pancreatic cells of the invention can be administered includes any abnormality or impairment in insulin production, e.g., expression and/or transport through cellular organelles, such as insulin deficiency resulting from, for example, loss of β cells as in IDDM (Type I diabetes), secretion, such as impairment of insulin secretory responses as in NIDDM (Type II diabetes), form of the insulin molecule itself, e.g., primary, secondary or tertiary structure, effects of insulin on target cells, e.g., insulin-resistance in bodily tissues, e.g., peripheral tissues, and responses of target cells to insulin. See Braunwald, E. et al. eds. Harrison's Principles of Internal Medicine, Eleventh Edition (McGraw-Hill Book Company, New York, 1987) pp. 1778-1797; Robbins, S. L. et al. Pathologic Basis of Disease, 3rd Edition (W. B. Saunders Company, Philadelphia, 1984) p. 972 for further descriptions of abnormal insulin activity in IDDM and NIDDM and other forms of diabetes. Administration of pancreatic cells of the invention to a recipient subject results in a reduction or alleviation of at least one adverse affect or symptom of a pancreatic disorder.

In further embodiment, neural cells obtained from a source (such as an abortus or a non-human animal) can be isolated by enzyme treatment and by tritrations through pipettes of decreasing diameter until a cell suspension is obtained. The cells can be genetically modified to express at least one immunoregulatory molecule prior to administering the cells to the desired area of the brain or the cells can be modified prior to isolation by obtaining the cells from a transgenic animal which contains neural cells expressing an immunoregulatory molecule. A common method of administrating cells into the brain of a recipient subject is by direct stereotaxic injection of the cells into the desired area of the brain. See e.g., Björklund, A. et al. (1983) Acta Physiol. Scand. Suppl. 522:1-75. The neural cells can be administered in a pharmaceutically acceptable carrier or diluent as described herein. To treat neurological deficits due to unilateral neurodegeneration in the brain of a human subject, about 12-24 million neural cells of the invention are introduced into the area of neurodegeneration. In humans with areas of brain neurodegeneration which occur bilaterally, about 12-24 million neural cells of the invention are introduced into each area of neurodegeneration, requiring a total of about 24-40 million neural cells.

The neural cells of the invention are particularly useful for the treatment of human subjects displaying neurodegenerative disorders which cause neurological deficits in the brain. Such brain neurodegeneration can be the result of disease, injury, and/or aging. As used herein, neurodegeneration includes morphological and/or functional abnormality of a neural cell or a population of neural cells. Non-limiting examples of morphological and functional abnormalities include physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or transmission of electrical impulses in abnormal patterns or at abnormal times. Neurodegeneration or neural injury can occur in any area of the brain of a subject and is seen with many disorders including, for example, head trauma, stroke, epilepsy, amyotrophic lateral sclerosis, pain, or multiple sclerosis, Huntington's disease, Parkinson's disease, and Alzheimer's disease.

In yet another embodiment, muscle cells can be obtained from a donor (e.g., by biopsy of a living related donor, from a brain dead human donor or from a transgenic animal containing muscle cells which express an immunoregulatory molecule) using a 14-16 gauge cutting trochar into a 1-2 inch skin incision. The fresh muscle plug can be lightly digested to a single cell suspension using collagenase, trypsin and dispase at 37° C. If the cells are not obtained from a transgenic animal as described herein, they can then be genetically modified to express at least one immunoregulatory molecule. Muscle cells are injected intramuscularly into a recipient patient in need of an increased store of muscle, e.g., an elderly patient with severe muscle wasting, or injected into a muscle group of a patient afflicted with Becker's or Duchenne muscular dystrophy. Furthermore, the cells can be administered in a pharmaceutically acceptable carrier as described herein.

Cardiomyocytes or skeletal myoblasts can also be used in the claimed methods. For example, heart tissue obtained from a donor, e.g., a non-human animal, or myoblasts obtained from a muscle biopsy from a subject can be manually sheared and treated with enzyme in order to isolate cardiomyocytes for use in treating insufficient cardiac function. The cardiomyocytes can be isolated from the heart of a transgenic animal which expresses an immunoregulatory molecule or can be genetically modified to express an immunoregulatory molecule after isolation of the cells as described herein. The period of viability of the cells after administration to a subject can be as short as a few hours, e.g., twenty-four hours, to a few days, to as long as a few weeks to months. One method that can be used to deliver the cardiomyocytes of the invention to a subject is direct injection of the cardiomyocytes into the ventricular myocardium of the subject. See e.g., Soonpaa, M. H. et al. (1994) Science 264:98-101; Koh, G. Y. et al. (1993) Am. J. Physiol. 33:H1727-1733. Cardiomyocytes can be administered in a pharmaceutically acceptable carrier as described herein. If cells are harvested from a pig for use in a human having a disorder characterized by insufficient cardiac function, about 10⁶-10⁷ pig cardiomyocytes can be introduced into the human, e.g., into the human heart.

The cardiomyocytes of the invention can be administered to a subject in order to reduce or alleviate at least one adverse effect or symptom of a disorder characterized by insufficient cardiac function. Adverse effects or symptoms of cardiac disorders are numerous and well-characterized. Non-limiting examples of adverse effects or symptoms of cardiac disorders include: dyspnea, chest pain, palpitations, dizziness, syncope, edema, cyanosis, pallor, fatigue, and death. For additional examples of adverse effects or symptoms of a wide variety of cardiac disorders, see Robbins, S. L. et al. Pathological Basis of Disease (W. B. Saunders Company, Philadelphia, 1984) pp. 547-609; Schroeder, S. A. et al. eds. Current Medical Diagnosis & Treatment (Appleton & Lange, Connecticut, 1992) pp. 257-356.

In addition, RPE cells or neural retina cells which express an immunoregulatory molecule can be used to treat retinal disorders. Neural retina cells and RPE cells obtained from a donor (e.g., a brain dead human donor or a non-human animal) can be disassociated from the eye cup using methods known in the art. See Edwards (1982) Methods Enzymology 81:39-43. Genetically modified neural retina cells or RPE cells which express an immunoregulatory molecule can be obtained from a transgenic animal or by other methods of genetic modification described herein. Neural retina cells and RPE cells are administered to a recipient subject by injecting the cells into the subretinal space of the subject. Common methods of administering cells into the subretinal space include, for example, the pars plana vitrectomy technique described in Lopez et al. (1987) Invest. Ophthamol. & Vis. Sci. 28:1131-1137, and Del Priore (1995) Arch. Ophthamol. 113:939-944; and, posterior transscleral approach as described by Durlu (1997) Cell Transplant. 6(2):149-162 and standard vitrepretinal surgery. The RPE cells or neural retina cells can be administered in a pharmaceutically acceptable carrier or diluent as described herein. To treat a human having a retinal disorder at least about 10⁵ to about 10⁶ RPE or neural retina cells are required.

Non-limiting examples of retinal disorders which RPE cells can be used include, for example, macular degeneration, retinitis pigmentosa, gyrate atrophy, fundus flavimaculatus, Stargardt's disease and Best's disease. Neural retina cells can be used for treatment of retinal disorders including, for example, retinitis pigmentosa, photoxic retinopathy and light damaged retina.

The cells used in these methods of the invention can be within a tissue or organ. Accordingly, in these embodiments, the tissue or organ is transplanted into the recipient subject by conventional techniques for transplantation. Acceptance of transplanted cells, tissues or organs can be determined morphologically or by assessment of the functional activity of the graft. For example, acceptance of liver cells can be determined by assessing albumin production, acceptance of pancreatic islet cells can be determined by measuring insulin production, and acceptance of neural cells can be determined by assessing neural cell function (e.g., production of dopamine by mesencephalic cells) or by measuring functional improvement in standardized tests (with parameters established prior to transplantation).

Administration of Genetically Modified Cells

The term “recipient subject” is intended to include mammals, preferably humans, in which an immune response is elicited against allogeneic or xenogeneic cells. A cell can be administered to a subject by any appropriate route which results in delivery of the cell to a desired location in the subject. For example, cells can be administered intravenously, subcutaneously, intramuscularly, intracerebrally, subcapsularly (e.g., under the kidney capsule) or intraperitoneally. The cells can be administered in a pharmaceutically acceptable carrier. A pharmaceutically acceptable carrier is a solution in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating cells genetically modified to express an immunoregulatory molecule, as described herein, in a pharmaceutically acceptable carrier, followed by filtered sterilization. Accordingly, one aspect of the invention features a composition comprising a cell which is genetically modified to express an immunoregulatory molecule capable of inhibiting T cell activation and/or a pharmaceutically acceptable carrier. In another embodiment, the composition can include both the genetically modified cells and exogenously added forms of one or both of the immunoregulatory molecules described herein.

Additional Treatment With Other Agents

Recipient subjects can further be treated with a T cell inhibitory agent according to the invention. Treatment can begin prior to, concurrent with or following transplantation of cells. The T cell inhibitory agent inhibits T cell activity. For example, the T cell inhibitory agent can be an immunosuppressive drug. A preferred immunosuppressive drug is cyclosporin A. Other immunosuppressive drugs which can be used include FK506 and RS-61443. An immunosuppressive drug is administered to a recipient subject at a dosage sufficient to achieve the desired therapeutic effect (e.g., inhibition of rejection of transplanted cells). Dosage ranges for immunosuppressive drugs, and other agents which can be coadministered therewith (e.g., steroids and chemotherapeutic agents), are known in the art (See e.g., Freed et al. (1992) New Engl. J. Med. 327:1549; Spencer et al. (1992) New Engl. J. Med. 327:1541; Widner et al. (1992) New Engl. J. Med. 327:1556; Lindvall et al. (1992) Ann. Neurol. 31:155; and Lindvall et al. (1992) Arch. Neurol. 46:615). A preferred dosage range for immunosuppressive drugs, suitable for treatment of humans, is about 1-30 mg/kg of body weight per day. A preferred dosage range for cyclosporin A is about 1-10 mg/kg of body weight per day, more preferably about 1-5 mg/kg of body weight per day. Dosages can be adjusted to maintain an optimal level of the immunosuppressive drug in the serum of the recipient subject. For example, dosages can be adjusted to maintain a preferred serum level for cyclosporin A in a human subject of about 100-200 ng/ml. It is to be noted that dosage values may vary according to factors such as the disease state, age, sex, and weight of the individual. Dosage regimens may be adjusted over time to provide the optimum therapeutic response according to the individual need and the professional judgment of the person administering or supervising the administration of the compositions, and that the dosage ranges set forth herein are exemplary only and are not intended to limit the scope or practice of the claimed compositions.

In one embodiment of the invention, an immunosuppressive drug is administered to a subject transiently for a sufficient time to induce tolerance to the transplanted cells in the subject. Transient administration of an immunosuppressive drug has been found to induce long-term graft-specific tolerance in a graft recipient (See Brunson et al. (1991) Transplantation 52:545; Hutchinson et al. (1981) Transplantation 32:210; Green et al. (1979) Lancet 2:123; Hall et al. (1985) J. Exp. Med. 162:1683). Administration of the drug to the subject can begin prior to transplantation of the cells into the subject. For example, initiation of drug administration can be a few days (e.g., one to three days) before transplantation. Alternatively, drug administration can begin the day of transplantation or a few days (generally not more than three days) after transplantation. Administration of the drug is continued for sufficient time to induce donor cell-specific tolerance in the recipient such that donor cells will continue to be accepted by the recipient when drug administration ceases. For example, the drug can be administered for as short as three days or as long as three months following transplantation. Typically, the drug is administered for at least one week but not more than one month following transplantation. Induction of tolerance to the transplanted cells in a subject is indicated by the continued acceptance of the transplanted cells after administration of the immunosuppressive drug has ceased. Acceptance of transplanted tissue can be determined morphologically (e.g., with biopsies of liver) or by assessment of the functional activity of the graft.

Alternatively, the T cell inhibitory agent can be one or more antibodies which deplete T cell activity, such as antibodies directed against T cell surface molecules (e.g., anti-CD2, anti-CD3, anti-CD4 and/or anti-CD8 antibodies). Antibodies are preferably administered intravenously in a pharmaceutically acceptable carrier or diluent (e.g., a sterile saline solution). Antibody administration can begin prior to transplantation (e.g., one to five days prior to transplantation) and can continue on a daily basis after transplantation to achieve the desired effect (e.g., up to fourteen days after transplantation). A preferred dosage range for administration of an antibody to a human subject is about 0.1-0.3 mg/kg of body weight per day. Alternatively, a single high dose of antibody (e.g., a bolus at a dosage of about 10 mg/kg of body weight) can be administered to a human subject on the day of introduction of the cells into the subject. The effectiveness of antibody treatment in depleting T cells from the peripheral blood can be determined by comparing T cell counts in blood samples taken from the subject before and after antibody treatment.

In another embodiment, the instant methods can further comprise treatment with a soluble form of an immunoregulatory molecule.

Dosage regimes for these additional agents can be adjusted over time to provide the optimum therapeutic response according to the individual need and the professional judgment of the person administering or supervising the administration of the compositions. Dosage ranges set forth herein are exemplary only and are not intended to limit the scope or practice of the claimed composition.

This invention is further illustrated by the following examples which should not be construed as limiting. The contents of all references and published patents and patent applications cited throughout the application are incorporated herein by reference.

### EXAMPLES

### Example 1

Elucidation of the mechanism of the immune response against transplanted porcine tissue is critical for the success of xenografting in humans. Both human T cells and NK cells recognize MHC antigens and human receptors may bind to MHC antigens across species barriers. Molecular characterization of porcine MHC class I clones from two MHC class I loci (P1 and P14) obtained from homozygous inbred miniature swine of three haplotypes (aa, cc, and dd), revealed extensive conservation between loci, suggesting that the genes were products of duplication from a common ancestral sequence. The level of homology between loci was similar to that between the haplotypes at each locus, suggesting that intergenic exchange had limited divergence of these genes. Comparison of the alleles indicated that the polymorphism occurred in the alpha-1 and alpha-2 domains of the class I heavy chain while the alpha-3 domain was highly conserved among the six genes analyzed. Amino acids in the alpha-2 and alpha-3 domains responsible for the binding of human CD8 to MHC class I were largely conserved in the porcine genes, but several critical residues were altered. Comparison of sequences recognized by human NK cell inhibitory receptors revealed that the residues critical for recognition by these receptors were altered in the porcine genes; thus the porcine class I molecules would be unable to inhibit lysis by human NK clones characterized to date. This finding provides a likely explanation for the susceptibility of porcine cells to cytolysis by human NK cells.

The understanding of the human immune response to porcine tissue has become increasingly important due to the development of clinical use of porcine tissue in transplantation (Sachs et al. 1976 Transplantation 22:559; Sachs 1994 Pathol. Biol. 42:217). The degree of homology between porcine and human transplantation antigens in combination with the cross-reactivity of adhesion and costimulatory molecules are likely to dictate how human T cells respond to the porcine tissue, as direct recognition of the MHC antigens will occur if the homology among these molecules is sufficient (Auchincloss 1990 Transplant Rev. 4:14; Auchincloss et al. 1993 Proc. Natl. Acad. Sci. USA 90:3373; Moses et al. 1990 J. Exp. Med. 172:567). Recent work has demonstrated MHC restriction of human T cells in their recognition of porcine cells: T cells reactive with a single haplotype of porcine MHC (termed SLA) were cloned after exposure to porcine tissue (Yamada et al. 1995 J. Immunol. 155:5249). Several recent studies have shown that human T cells can recognize porcine MHC molecules directly (Murray et al. 1994 Immunity 1:57; Rollins et al. 1994 Transplantation 57:1709; Yamada et al. 1995 J. Immunol. 155:5249) and that this recognition can lead to killing of porcine cells (Yamada et al. 1995 J. Immunol. 155:5249). Porcine cells have recently been shown, moreover, to be targets for human NK cells (Donnelly et al. 1997 Cells Immunol. 175:171; Seebach et al. 1996 Xenotransplantation 3:188). As human MHC class I molecules deliver a negative signal to human NK cells that protects syngeneic cells from lysis (Gumperz et al. 1995 Nature 378:245; Raulet et al. 1995 Cell 82:697), alterations in the sequence of the porcine MHC class I genes could be responsible for cytolysis of porcine cells due to a lack of recognition by human NK cell receptors.

With the availability of pigs inbred at the MHC, it should be possible to address these questions. Characterization of the MHC class II genes from these animals has revealed homology between porcine and human DRB genes (Gustafsson et al. 1990 Proc. Natl. Acad. Sci. USA 87:9798). Although early studies established the presence of seven porcine class I genes and reported the genomic sequence of two such genes, these sequences were both obtained from the dd haplotype (Singer et al. 1982 Proc. Natl. Acad. Sci. USA 79:1403; Singer et al. 1987 Vet. Immunol. Immunopath. 17:211; Satz et al. 1985 J. Immunol. 135-2167).

The sequence of three haplotypes of two MHC class I genes from inbred miniature swine has been determined and a high degree of homology between the two loci has been demonstrated. The three alleles of each locus are polymorphic in the peptide binding regions of the alpha-1 and alpha-2 domains, but the sequence of the alpha-3 domain is conserved. The sequence data indicates that the consensus motifs for binding of human NK cell receptors are largely lacking in the porcine genes. In addition, sequences for binding of CD8 that are conserved among human MHC class I haplotypes are not completely conserved in the porcine class I sequences. These findings lead to an expectation of a decreased strength of the interaction of human T cells with porcine as compared to allogeneic targets and are consistent with the finding that human NK cells appear to kill porcine cells.

Materials and Methods

Isolation and sequencing of porcine MHC class I cDNA—Total RNA was isolated from either porcine smooth muscle cells (aa and dd haplotype miniature swine) or from porcine peripheral blood lymphocytes (cc haplotype) using RNAzol B following the manufacturer's protocol (Tel-Test, Inc.). The first strand of cDNA was generated using 1 ug of total RNA primed with oligo dT by reverse transcription (Clontech 1st-Strand cDNA Synthesis Kit). PCR was carried out using 5′ primers designed from the genomic sequence for PD1 and PD14 (Satz et al. 1985 J. Immunol. 135:2167) with restriction sites for Hind III and Xho I indicated: ATCGAAGCTTATGGGGCCTGGAGCCCTCTTCCTG for the 5′ primer of the P1 genes and ATCGAAGCTTATGCGGGTCAGAGGCCCTCAAGCCATCCTCATTC for the 5′ primer for the P14 genes. The 3′ primer for both cDNAs was CGATCTCGAGTCACACTCTAGGATCCTTGGGTAAGGGAC. PCR was performed by a “touchdown” (Don et al. 1991 Nucleic Acids Res. 19:4008; Roux 1994 Biotechniques 16:812) method in which denaturation was carried out at 94° C., and annealing was performed at temperatures ranging from 72° C. to 60° C. for 1 min with 2 cycles at each temperature followed by 10 cycles at 60° C. PCR products were cloned into pGem7Zf (+) (Promega) for sequencing using Sequenase Version 2.0 (USB). Both strands of DNA were sequenced. Multiple PCR reactions were performed to obtain independent clones for each gene, and at least two clones corresponding to each gene were sequenced for confirmation of the reported sequences.

Restriction digest analysis—The SLA cDNA clones were analyzed by restriction mapping as follows: 1 ug of DNA (SLA clone in pGem7Zf) was digested with Hind III and Xho I at 37° C. for 2 hours or with BsmB I at 55° C. for 2 hours. Products were separated on 1% agarose gels (Gibco) and stained with ethidium bromide.

Transfection—The class I genes were inserted at Hind III/Xba I sites into pcDNA3 (Invitrogen) which was modified to contain a thymidine kinase promoter. The mouse lymphoma cell line C1498 (H-2b) was utilized. Electroporation was carried out at 270 V, 960 uF using 50 ug DNA and 107 cells in serum free RPMI medium. Cells were grown in DMEM containing 10% fetal calf serum and were selected beginning 48 h after transfection in 800 ug/ml G418. Media was changed every two days and after three weeks, PD1 transfected cells were selected with anti-mouse IgG conjugated magnetic beads (Dynal) coated with anti-SLA antibody 9-3 (Oettinger et al. 1996 Xenotransplantation In press). Two weeks later these cells underwent a second round of magnetic bead selection. This cell population was cloned by limiting dilution into 96 well plates. Control cells were transfected with vector alone. Positive PD1 and PD14 expressing clones were screened by flow cytometry analysis with a FACScan (Becton Dickinson) using anti-SLA antibodies, PT-85 (VMRD) and 9-3 (Oettinger et al. 1996 Xenotransplantation In press) at a concentration of 1 μg/2×10⁵ cells. Fluorescein-conjugated donkey anti-mouse IgG (Jackson) was added for detection. Cells were incubated with antibody for 1 h at 4° C. in PBS containing 0.5% BSA and after addition of secondary antibody were further incubated for 30 m at 4° C. As a control for H-2b expression, the cells were tested with anti-H-2 antibody, M1/42.

Results

Isolation of MHC class I genes from homozygous aa, cc or dd pigs—RNA isolated from inbred miniature swine of three haplotypes was reverse transcribed and amplified employing primers for P1 and P14 genes. Six cDNAs were obtained (a P1 and P14 product from each haplotype), and the cDNAs were compared by digestion with restriction enzymes. The distinct patterns obtained for the products derived from P1 and P14 specific primers indicated that we had obtained clones corresponding to the P1 and P14 loci from each of the three haplotypes, and we therefore designated the genes by their locus and haplotype as PA1, PC1, PD1, and PA14, PC14 and PD14. The successful reverse transcription demonstrated that both genes were expressed in porcine cells.

Sequence homology among six porcine MHC class I genes—Within each locus the cDNA sequences of the three haplotypes displayed a high degree of homology (The sequence data are available from EMBL/GenBAnk/DDB under accession numbers AF01 4001, AF01 4002, AF01 4003, AF01 4004, AF01 4005, and AF01 4006). Comparison of the pairs of haplotypes within P1 indicated an average of 55 nucleotide differences out of 1086 bases with a range of 31-67 differences. A similar comparison at the P14 locus yielded an average of 64 differences with a range of 43-80. Comparison of pairs of HLA alleles within a much larger sample of HLA-A, B and C loci gave an average value of 35 differences with a range of 1-85 (Parham et al. 1995 Immunol. Rev. 143:141).

Homology between the two loci was of a similar magnitude. Comparison of each pair of P1 and P14 genes yielded an average of 68 nucleotide differences between the loci with a range of 52-79. This compares with an average of 10⁴ differences and a range of 55-141 found for HLA genes (Parham et al. 1995 Immunol. Rev. 143:141).

The deduced amino acid sequence of the two loci indicated that the extensive homology observed among the haplotypes of each locus was also evident between the two loci. All six genes shared considerable sequence, particularly in the alpha-3 domain and transmembrane and cytoplasmic regions. P14 contained three additional amino acids at the N-terminus of the leader sequence that confirmed the identity of the three genes as P14 alleles (Satz et al. 1985 J. Immunol. 135:2167).

Expression of porcine MHC class I on the cell surface of mouse lymphoblasts. The cDNAs for two of the MHC class I genes were transfected into mouse cell lines to determine whether the clones we had obtained would be expressed. In each case expression could be seen as detected with an antibody, 9-3, against a monomorphic determinant in the alpha-3 domain of the MHC class I molecule. An antibody, PT-85, against a determinant on SLA that is dependent on the conformation of the class I molecule, reacted with both the PD1 and PD14 gene products expressed in the C1498 cells as measured by FACS.

Sites of polymorphism in the porcine class I genes—The polymorphic sites in the porcine class I genes were analyzed by variability plots of the individual sequences. The plots showed that the greatest degree of polymorphism were within the alpha-1 and alpha-2 domains. The alpha-3 domains differed by a single amino acid in one haplotype. In the alpha-1 domain, the sites of greatest polymorphism corresponded to those seen in the human genes and correlated with the portions of the alpha helix that face the antigen binding groove of the MHC class I molecule; the sites of polymorphism in the alpha-1 domain were clustered at positions 62-79. However, unlike the human genes in which the sites of polymorphism in the alpha-2 domain are predominantly in the β-pleated sheets (Parham et al. 1988 Proc. Natl. Acad. Sci. USA 85:4005), in the SLA genes the regions of greatest polymorphism were in the alpha helical portion of the alpha-2 domain. In the alpha-2 domain, the sites with greatest variability were at positions 156 and 163; the positions that displayed the greatest polymorphism in the alpha-2 domain of HLA (Parham et al. 1988 Proc. Natl. Acad. Sci. USA 85:4005), 95, 97, 114 and 116, displayed less variability in SLA.

Two additional sites of homology between the porcine and human sequences were conserved among all six genes. The cysteines at positions 101 and 164 and those at 203 and 259 form disulfide bonds in HLA and were present in the porcine sequences. The N-linked glycosylation consensus sequence at positions 86-88 was conserved in all six genes.

Analysis of consensus sequences for recognition of MHC class I by human T cells and NK cells—The human T cell response against porcine tissue has been shown to occur largely through direct recognition of porcine antigen presenting cells by the human T cell (Murray et al. 1994 Immnunity 1:57; Rollins et al. 1994 Transplantation 57:1709; Yamada et al. 1995 J. Immunol. 155:5249), as well as through an indirect mechanism in which porcine antigens are processed and presented to human T cells by human antigen presenting cells (Yamada et al. 1995 J. Immunol. 155:5249). This implies that the human T cell receptor can recognize porcine MHC, and human T cells that can kill porcine cells have been demonstrated (Donnelly et al. 1997 Cell. Immunol. 175:171; Yamada et al. 1995 J. Immunol. 155:5249). An interaction of CD8 molecules on the T cell surface with MHC class I on the target increases the strength of the effector function. Comparison of sequences required for binding of human CD8 to human MHC class I (Salter et al. 1990 Nature 345:41) to the sequences present in the porcine MHC genes which have been characterized indicated that at least two of the amino acids in the primary binding site were altered: one of these changes (Thr 225->Ser 225)was conservative but a second (Thr 228->Met 228) was nonconservative and may therefore result in a decresed affinity interaction of human T cells with porcine MHC class I.

Porcine cells have recently been shown to be susceptible to lysis by human NK cells. NK clones are known to be inhibited by MHC class I in the autologous situation, and recent studies have elucidated sequences present in MHC class I that are recognized by specific receptors on human NK cells and account for resistance to lysis (Gumperz et al. 1995 J. Exp. Med. 181:1133; Colonna et al. 1993 Proc. Natl. Acad. Sci. 90:12000; Biassoni et al. 1995 J. Exp. Med. 182:605; Cella et al. 1994 J. Exp. Med. 180:1235). shows a comparison of the known sequences that confer resistance to human NK receptors to the sequences found in the porcine MHC class I molecules; for the group 1 clones, Lys 80 is the key residue conferring resistance, whereas for group 2, Ser 77 (Biassoni et al. 1995 J. Exp. Med. 182:605) and Asn 80 (Mandelboim et al. 1996 J. Exp. Med. 184:913) have both been implicated as the critical amino acid. For HLA-B an Ile at position 80 accounts for binding of the NKB1 receptor and prevents lysis by NK cells that express this receptor (Cella et al. 1994 J. Exp. Med. 180:1235). In addition, recently reported inhibitory receptors that recognize HLA-A may be inhibited by Asp at position 74 (Dohring et al. 1996 J. Immunol. 156:3098; Storkus et al. 1991 Proc. Natl. Acad. Sci. USA 88:5989), and this residue was not found in the porcine class I sequence. None of the sequences that these negative receptors recognize were present in the porcine molecules characterized in this study except for Asn at position 80 in PC1.

Discussion

Porcine MHC class I genes derived from three haplotypes of inbred miniature swine have been characterized. This information has provided insight into the potential for interactions between the human immune system and porcine antigen presenting cells. The recognition of tissue grafts across the pig to human species barrier is dependent on both direct and indirect recognition of porcine MHC by human T cells (Yamada et al. 1995 J. Immunol. 155:5249; Rollins et al. 1994 Transplantation 57:1709; Murray et al. 1994 Immunity 1:57). The use of pigs inbred at MHC has allowed the isolation of haplotypes defined by polymorphisms in the MHC genes (Sachs et al. 1976 Transplantation 22:559), but the molecular characterization of the class I haplotypes has not previously been reported. Understanding of MHC restriction in xenotransplantation will be advanced by characterization of gene polymorphism, as recent data has shown that human T cells specific for porcine targets appear to recognize the MHC haplotype of the target cell (Yamada et al. 1995 J. Immunol. 155:5249). The data presented here is the first information at a molecular level on the inbred MHC class I haplotypes recognized by these recipient T cells.

The high degree of homology between P1 and P14 indicates that these two loci are likely to be products of gene duplication from a common ancestral sequence; in addition, genetic exchange between the two loci may account for the conservation of sequence. Changes within the alleles of each locus may have arisen from independent mutational events as it is thought that new sequences within the peptide binding regions of the class I molecule are favored in evolution due to the selective advantage conferred by the ability to present peptides from novel pathogens (Parham et al. 1995 Immunol. Rev. 143:141). However fixation of random mutations appears to have been infrequent in the evolution of class I genes, and the major mechanism for generation of new alleles of human MHC class I genes has been gene conversion resulting from exchange between alleles within a locus. Genetic exchange between loci has been infrequent relative to exchange within loci for human MHC class I genes but is a major factor in the production of new alleles in mouse class I genes (Pease et al. 1991 Crit. Rev. Immunol. 11:1). The high degree of homology between the P1 and P14 loci (average of 68 differences between pairs as compared to 104 differences (Parham et al. 1995 Immunol. Rev. 143:141) in human class I genes) indicates that they may have formed new alleles by intergenic exchange as in the mouse.

The sites of polymorphism among MHC class I genes from inbred pigs of different haplotypes revealed that the polymorphisms occurred in areas of the gene analogous to those seen in human MHC class I (Parham et al. 1988 Proc. Natl. Acad. Sci. USA 85:4005). The alpha-1 and alpha-2 subunits of swine MHC class I contained almost all of the polymorphic sites, and within these subunits the variability was concentrated in several hypervariable regions. In the alpha-1 subunit these areas were between amino acids 62 and 79. These regions in the alpha-1 domain of SLA are analogous to the regions in HLA that contain the highest degree of heterogeneity based on a comparison of 39 haplotypes of HLA-A, -B and -C (Parham et al. 1988 Proc. Natl. Acad. Sci. USA 85:4005). In the alpha-2 subunit the region of major variability based on our limited sample was between residues 152 and 167 which is the corresponding alpha-helical region of the alpha-2 domain. The sites of greatest variability were positions 156 and 163; this contrasts with HLA which displays heterogeneity at these two positions but is most polymorphic in the beta-strand (residues 95-116).

The sequences reported here for PD1 and PD14 differed at a number of bases from the sequences reported by Singer et al. (Singer et al. 1982 Proc. Natl. Acad. Sci. USA 79:1403; Singer et al. 1987 Vet. Immunol. Immunopath. 17:211; Satz et al. 1985 J. Immunol. 135:2167). The reason for the discrepancies are not certain but could be due to related sequences that are non identical but share considerable sequence homology. For example, using the primers for PCR amplification of P14, closely related genes were obtained from the cc haplotype pigs that differed from PC14 by 20 single nucleotide changes, indicating that another class I gene may be transcribed from the pig genome. This comparison resolves the question raised on the basis of the genomic sequences (Satz et al. 1985 J. Immunol. 135:2167) as to the heterogeneity in the alpha-1 and alpha-2 sequences. Both domains contained considerable heterogeneity in the regions in which polymorphisms are seen in the human and mouse sequences. Our data indicated that both genes were expressed in normal porcine cells as we were able to obtain the mRNAs for all six of the genes that we sequenced. Comparison of our deduced amino acid sequences to previously reported N-terminal sequences of SLA purified from the same three haplotypes of miniature swine (Metzger et al. 1982 J. Immunol. 129:716) also indicated that both loci were expressed: the amino acid sequences reported for the d and c haplotypes were identical to the PD14 and PC14 sequences reported here, whereas the amino acid sequence reported for the a haplotype was evidently a mixture of two proteins. Some residues from the reported sequence match our PA1 sequence and others correspond to PA14.

Several recent studies on the human anti-pig response have shown that human NK cells can kill porcine cells (Seebach et al. 1996 Xenotransplantation 3:188; Donnelly et al. 1996 175:171) and have raised the question of the targets recognized on porcine cells. Other investigators have shown that NK cells are regulated in part by receptors for MHC class I (Cella et al. 1994. J. Exp. Med. 180:1235; Raulet et al. 1995 Cell 82:697; Gumperz et al. 1995 J. Exp. Med. 181:1133; Colonna et al. 1993 Proc. Natl. Acad. Sci. 90:12000; Gumperz et al. 1995 Nature 378:245; Biassoni et al. 1995 J. Exp. Med 182:605). These receptors are thought to deliver a negative signal to NK cells, such that cells bearing MHC class I molecules recognized by an inhibitory receptor on an NK cell are protected from cytolysis. Porcine cells might lack such a signal or, alternatively, porcine MHC molecules or other ligands may be recognized by NK receptors that transmit a positive signal for NK mediated killing (Bezouska et al. 1994 Nature 372:150). The sequences in HLA known to inhibit cytotoxicity by the NK clones characterized to date were not present in the porcine MHC class I genes with the exception of PC1. The absence of sequences known to be important for recognition by NK receptors therefore suggests that porcine cells are susceptible to killing by human NK cells due to the absence of a negative signal. The PC1 protein contains an Asn at position 80 and would confer resistance to human group 2 NK cells according to a recent study (Mandelboim et al. 1996 J. Exp. Med. 184:913), although a previous report had indicated that Ser at position 77 was the key residue for inhibition of group 2 clones (Biassoni et al. 1995 Nature J. Exp. Med. 182:605).

The binding sites for human CD8 on HLA have been localized to three areas in he alpha-3 domain (Salter et al. 1990 Nature 345:41) and more recently to a face of the alpha helix in the alpha-2 domain (Sun et al. 1995 J. Exp. Med. 182:1275). Most of these sites were partially conserved in the porcine MHC class I molecule. The SLA genes showed complete agreement of the three residues (Gln 115, Asp 122 and Glu 128) in the alpha-2 domain identified as critical for the binding of CD8 to human MHC class I and shared homology at most of the critical sites in the alpha-3 domain. Two of these sites had conservative changes in the pig genes, a Thr→Ser change at position 225 and a Val→Leu change at position 247. However, all six of the genes sequenced here coded for Met at position 228 in contrast to human MHC class I which has a conserved Thr at that position. Mutation of this residue to Ala resulted in a loss of CD8 binding and reduction in the cytotoxic activity by CTL clones that recognize MHC class I (Parham et al. 1988 Proc. Natl. Acad. Sci. USA 85:4005). Therefore an altered affinity of human CD8 for porcine MHC class I as compared to human would be expected. In mouse targets, amino acid changes in the alpha-3 domain that weaken the interaction of the target with CD8 have been shown to result in an attenuated response in vitro (Sekimata et al. 1993 J. Immunol. 150:4416; Newberg et al. 1992 J. Immunol. 149:136; Kalinke et al. 1990 Nature 348:642; Irwin et al. 1989 J. Exp. Med. 170:1091). Experiments using transgenic mice that express HLA have shown that CTLs have enhanced activity toward a chimeric class I molecule with a mouse alpha-3 domain and human alpha-1 and alpha-2 domains (Sekimata et al. 1993 J. Immunol. 150:4416; Newberg et al. 1992 J. Immunol. 149:136; Kalinke et al. 1990 Nature 348:642; Irwin et al. 1989 J. Exp. Med. 170:1091). Other investigators have observed that a human alpha-3 domain weakens the murine cytotoxic T cell response toward mouse targets (Sekimata et al. 1993 J. Immunol. 150:4416; Newberg et al. 1992 J. Immunol. 149:136; Kalinke et al. 1990 Nature 348:642; Irwin et al. 1989 J. Exp. Med. 170:1091). The H-2kb alpha-3 domain has a Met at position 228 and a Leu in place of Gln at position 224. These two changes are thought to weaken human CD8 binding to the mouse alpha-3 domain. The sequence of the porcine genes at this site would be expected to confer a higher affinity for human CD8 than that of mouse MHC class I but a lower affinity than human MHC class I.

A decreased affinity of CD8 for MHC class I would affect the CTL response to porcine targets. Numerous studies have shown that the interaction between a CTL and its target is strengthened by the binding of the CD8 coreceptor to MHC class I (Luescher et al. 1995 Nature 373:353; Kane et al. 1993 J. Immunol. 150:4788). The CD8 molecule has a binding site for p561ck on its cytoplasmic domain and is thought upon engagement to augment the signal sent to the T cell (O'Rourke et al. 1994 J. Immunol. 4359; Kane et al. 1993 J. Immunol. 150:4788). In the absence of this interaction T cells have been shown to react less strongly (Geppert et al. 1992 Eur. J. Immunol. 22:1379). Thus, the changes found here at a molecular level are likely to influence the cellular interactions that govern the immune response.

Responses between a number of xenogeneic pairs are thought to occur in an indirect manner via presentation of processed foreign antigens on the surface of host antigen presenting cells. In the absence of a direct interaction, the affinity of CD8 for MHC class I would be irrelevant. Several recent studies have concluded that the human anti-porcine immune response can be direct (Murray et al. 1994 Immunity 1:57; Rollins et al. 1994 Transplantation 57:1709; Yamada et al. 1995 J. Immunol. 155:5249), and, therefore, the affinity of CD8 for porcine MHC class I could play a role in regulating the strength of the human immune response to porcine tissue. The strength of the human anti-porcine response is likely to be determined by a number of factors, but a weakened interaction of cytotoxic T cells with porcine targets would be expected to permit immunosuppression of this arm of the response using therapy capable of inhibiting a human allogeneic response.

The contents of Sullivan et al. (1997) J. Immunol. 159(5):2318-2326 are hereby incorporated by reference.

### Example 2

### Transplantation of Hepatocytes Expressing Human Fas Ligand

FasL Construct

The gene encoding human FasL (the nucleotide sequence of which is provided in Takahashi et al. (1994) Int. Immunol. 6(10): 1567-1574) is ligated to an albumin promoter for liver specific expression. The FasL/promoter is inserted into pcDNA3 (Invitrogen, San Diego, Calif.) which is modified with splicing and polyadenylation sites provided by a fragment of α-globin gene including two exons and an intron with 400 bp of 3′ untranslated region spliced into the 3′ end of the FasL gene.

The FasL construct is excised from the pcDNA3 expression vector with restriction enzymes and then purified by agarose gel electrophoresis.

Production of Transgenic Pig

The purified human FasL DNA construct is introduced into the pronuclei of a fertilized oocyte by microinjection as described in detail herein and in Hogan, B. et al., A Laboratory Manual (Cold Spring Harbor Laboratory Press, Cold Spring Harbor, N.Y., 1986). The oocyte is then allowed to develop in a pseudopregnant female foster pig. The foster pig is allowed to carry the fetuses to term.

Upon birth of the litter, the tissues of the transgenic pigs are analyzed for the presence of FasL by either directly analyzing RNA, assaying the tissue for FasL, or by assaying conditioned medium for secreted FasL. For example, in vitro techniques for detection of FasL mRNA include Northern hybridizations and in situ hybridizations. In vitro techniques for detection of FasL protein include enzyme linked immunosorbent assays (ELISAs), Western blots, immunoprecipitations and immunofluorescence. In vitro techniques for detection of FasL genomic DNA include Southern hybridizations. Furthermore, in vivo techniques for detection of FasL protein include introducing into a subject a labeled anti-FasL antibody. For example, the antibody can be labeled with a radioactive marker whose presence and location in a subject can be detected by standard imaging techniques.

Isolation and Transplantation of Hepatocytes Expressing FasL

Porcine hepatocytes are isolated by the two stage perfusion technique originally described by Berry and Friend ((1969) J. Cell Biol. 43:506-520) and modified by others (Maganto P. et al. (1992) Transplant Proc. 24:2826-2827; Gerlach J. C. et al. (1994) Transplantation 57:1318-1322) for ex vivo perfusion of large animal organs and described in detail in PCT Publication Number WO 96/37602 published on Nov. 28, 1996. A liver lobe of 100-200 g is cannulated and perfused with HBSS (minus Mg⁺⁺, Ca⁺⁺) containing 0.4 mM EDTA, 10 mM HEPES, pH 7.4 and penicillin (100 U/ml)-streptomycin (100 ug/ml) at 35° C. This is followed by a second perfusion with complete HBSS containing collagenase P (0.8 mg/ml, Boehringer Mannheim), 10 mM HEPES, pH 7.4, and penicillin-streptomycin at 35° C. The perfusion is continued until visible softening of the organ occurs. The total time for digestion ranges from 12-20 minutes. The digested liver is then physically disrupted and the released hepatocytes are washed (50×g) twice in DMEM/Weymouth media containing 10% heat inactivated calf serum at 4° C.

Porcine hepatocytes are collected and counted. Viability is assessed by trypan blue staining. The purity of the hepatocyte preparation is judged by immunofluorescence for class II bearing non-parenchymal cells. Purity determinations are made by counting the positive staining cells (monoclonal antibody ISCR3) in several fields consisting of 200 cells.

The isolated porcine hepatocytes expressing FasL are transplanted by infusion into the splenic artery of a patient having chronic end-stage liver disease with acute decompensation or acute liver failure with pathologic verified diagnosis. Strom et al. (1997) Transplantation 63(4):559-569. Graft survival is assessed by measuring serum ammonia levels in the recipient as described in Strom et al., supra.

### Example 3

### Transplantation Of Porcine Mesencephalic Cells Expressing Human CD40

CD40 Construct

The human CD40 gene (the nucleotide sequence which is provided in Stamenlovic et al. (1988) EMBO J. 7:1053-1059) is fused to the constant domain and secretory signal of Ig by methods known in the art. The CD40/1 g fusion product having BamHI/XhoI restriction sites at the 5′ and 3′ ends is spliced into the pcDNA3 expression vector (Invitrogen, San Diego, Calif.) which is modified to contain a tyrosine hydroxylase promoter for expression within dopaminergic areas of the brain.

Production of Transgenic Pig

The DNA construct which encodes human CD40 is introduced into the pronuclei of a fertilized oocyte by microinjection as described in detail herein and in Hogan, B. et al., A Laboratory Manual (Cold Spring Harbor Laboratory Press, Cold Spring Harbor, N.Y., 1986). The oocyte is then allowed to develop in a pseudopregnant female foster pig. The foster pig is allowed to carry the fetuses to term.

Upon birth of the litter, the tissues of the transgenic pigs are analyzed for the presence of CD40 by either directly analyzing RNA, assaying the tissue for CD40, or by assaying conditioned medium for secreted CD40. For example, in vitro techniques for detection of CD40 mRNA include Northern hybridizations and in situ hybridizations. In vitro techniques for detection of CD40 protein include enzyme linked immunosorbent assays (ELISAs), Western blots, immunoprecipitations and immunofluorescence. In vitro techniques for detection of CD40 genomic DNA include Southern hybridizations. Furthermore, in vivo techniques for detection of CD40 protein include introducing into a subject a labeled anti-CD40 antibody. For example, the antibody can be labeled with a radioactive marker whose presence and location in a subject can be detected by standard imaging techniques.

Isolation and Transplantation of Ventral Mesencephalic Cells Expressing CD40

Ventral mesencephalic cells are isolated from transgenic pig brain by methods known in the art. For example, the ventral mesencephalic cells are isolated by the methods described in PCT Publication Number WO 96/14398 published on May 17, 1996. Briefly, the ventral mesencephalon (VM) is dissected from the surrounding tissue and collected in a petri dish containing Dulbecco's PBS. The VM fragments are incubated at 37° C. for 10 minutes in 1.5 ml of pre-warmed 0.05% Trypsin-0.53 mM EDTA (Sigma) in calcium- and magnesium-free Hanks Balanced Salt Solution (HBSS). The tissue is then washed four times with HBSS with 50 μg/ml Pulmozyme (human recombinant DNase, Genentech), and then gently triturated through a series of fire-polished Pasteur pipettes of decreasing diameter until a cell suspension containing single cells and small clumps of cells is obtained. Cell number and viability are determined under fluorescence microscopy using acridine orange-ethidium bromide as previously described. Brundin, P. et al. (1985) Exp. Brain Res. 60:204-208.

The isolated VM cells expressing CD40 are transplanted into the striatum of a Parkinson's patient by direct stereotaxic injection into the striatum. Assessment of graft survival is monitored by MRI and functional recovery is assessed by variations in the patient's Unified Parkinson's Disease Rating Scale (UPDRS) score.

### Example 4

### Transplantation Of Porcine Cortical Cells Expressing Human CD8

CD8 Construct

The human CD8 gene (the nucleotide sequence which is provided in Shuie (1988) J. Exp. Med. 168:1993-2005 and Nakayama (1989) ImmunoGenetics 30:393-397) is cloned into a pcDNA3 (Invitrogen, San Diego, Calif.) which contains a neomyocin resistance gene. The pcDNA3 vector is also modified to contain a H2k^{b} promoter for general expression in several tissue types including cortical cells. In addition, the pcDNA3 includes splice and polyadenylation sites.

Production of Transzenic Pig

The DNA construct which encodes human CD8 is introduced into the pronuclei of a fertilized oocyte by microinjection as described in detail herein and in Hogan, B. et al., A Laboratory Manual (Cold Spring Harbor Laboratory Press, Cold Spring Harbor, N.Y., 1986). The oocyte is then allowed to develop in a pseudopregnant female foster pig. The foster pig is allowed to carry the fetuses to term.

Upon birth of the litter, the tissues of the transgenic pigs are analyzed for the presence of CD8 by either directly analyzing RNA, assaying the tissue for CD8, or by assaying conditioned medium for secreted CD8. For example, in vitro techniques for detection of CD8 mRNA include Northern hybridizations and in situ hybridizations. In vitro techniques for detection of CD8 protein include enzyme linked immunosorbent assays (ELISAs), Western blots, immunoprecipitations and immunofluorescence. In vitro techniques for detection of CD8 genomic DNA include Southern hybridizations. Furthermore, in vivo techniques for detection of CD8 protein include introducing into a subject a labeled anti-CD8 antibody. For example, the antibody can be labeled with a radioactive marker whose presence and location in a subject can be detected by standard imaging techniques.

Isolation and Transplantation of Cortical Cells Expressing CD8

Cortical cells are isolated from transgenic pig brain by methods known in the art. For example, the cortical cells are isolated by the methods described in PCT Publication Number WO 96/14398 published on May 17, 1996. Briefly, the cortical anlage from the transgenic pig is dissected, taking care to remove only presumptive motor/somatosensory cortex and not limbic cortex.

Pig tissues is collected in sterile Hank's balanced salts solution (HBSS; Sigma Chemical Co., St. Louis, Mo.). The cortical tissue is incubated at 37° C. in 0.5% trypsin and DNase (80 Kunitz units/ml) for 30 minutes, washed three times with HBSS, and then carefully triturated with a fire-polished Pasteur pipette until homogenous suspensions are obtained. Cortical cell viability and concentration is determined by the acridine orange/ethidium bromide exclusion method as described in Brundin, P. et al. (1985) Brain Res. 331:251-259.

Each site of seizure of patients with focal epilepsy is identified by depth EEG electrode and the isolated cortical cells expressing CD8 are transplanted by direct stereotaxic injection into the tissue that has been determined by the specific depth electrode to lie within the site of seizure onset. Assessment of graft survival is monitored by MRI and functional recovery is assessed by variations in the patient's interval seizure history.

### Example 5

### Transplantation Of Porcine Pancreatic Islet Cells Expressing Human CD40 Ligand

CD40 Ligand Construct

The gene encoding human CD40 ligand (the nucleotide sequence of which is provided in Graf et al. (1992) Eur. J. Immunol. 22:3191-3194) is cloned into a pcDNA3 (Invitrogen, San Diego, Calif.) which contains a neomyocin resistance gene. The pcDNA3 vector is also modified to contain a H2k^{b} promoter for general expression in several tissue types including pancreatic islet cells. In addition, the pcDNA3 includes splice and polyadenylation sites.

Production of Transgenic Pig

The DNA construct which encodes human CD40 ligand is introduced into the pronuclei of a fertilized oocyte by microinjection as described in detail herein and in Hogan, B. et al., A Laboratory Manual (Cold Spring Harbor Laboratory Press, Cold Spring Harbor, N.Y., 1986). The oocyte is allowed to develop in a pseudopregnant female foster pig. The foster pig is allowed to carry the fetuses to term.

Upon birth of the litter, the tissues of the transgenic pigs are analyzed for the presence of CD40 ligand by either directly analyzing RNA, assaying the tissue for CD40 ligand, or by assaying conditioned medium for secreted CD40 ligand. For example, in vitro techniques for detection of CD40 ligand mRNA include Northern hybridizations and in situ hybridizations. In vitro techniques for detection of CD40 ligand protein include enzyme linked immunosorbent assays (ELISAs), Western blots, immunoprecipitations and immunofluorescence. In vitro techniques for detection of CD40 ligand genomic DNA include Southern hybridizations. Furthermore, in vivo techniques for detection of CD40 ligand protein include introducing into a subject a labeled anti-CD40 ligand antibody. For example, the antibody can be labeled with a radioactive marker whose presence and location in a subject can be detected by standard imaging techniques.

Isolation and Transplantation of Pancreatic Islets Expressing CD40 Ligand

Cells expressing CD40 ligand are isolated by methods known in the art. For example, pancreatic islet cells are isolated from the transgenic pig by the method described in PCT Publication Number WO 96/12794 published on Oct. 18, 1995. Briefly, solid pancreatic tissue samples are dissected from surrounding gut tissue, e.g., by dissecting the tissue under a dissecting microscope. The tissue is then resuspended in 1.5 ml of 0.05% Trypsin, 0.53 mM EDTA and incubated at 37° C. for 15 minutes. Tissue is dissociated by triturating with a pasteur pipette until a uniform cell suspension is formed. Trypsin is stopped by adding 5 ml of medium (RPMI-1640+10% FCS), then the cells are collected at 1000 RPM for 5 minutes at 25° C. Cells are resuspended in culture media (RPMI-1640+10% FCS+5 ng/ml PDGF+100 ng/ml EGF) and plated in sterile tissue culture dishes. Cells are then allowed to adhere and grow at 37° C. in an incubator with 5% CO₂.

Using a catheter, the islet cells are injected into the portal vein of a subject recipient, e.g., a human with diabetes as described in Andersson et al. (1992) Transplant. Proceed 24(2):677-678. The success of the islet transplantation is monitored by the detection of porcine C-peptide in the serum of the recipient. Andersson et al., supra.

### Example 6

### Transplantation Of Porcine Striatal Cells Expressing Human Fas Ligand And Modified Porcine MHC Class I Killer Inhibitory Sequence

A DNA construct encoding human FasL is prepared as described in Example I.

The nucleotide sequence encoding porcine MHC class I (e.g., PA14 locus) is modified by site directed mutagenesis to produce an MHC class I protein having an asparagine at position 77 and a lysine at position 80, the amino acid residues found to be critical for binding NK cells in humans via their inhibitory receptors (Sullivan et al. (1997) J. Immunol 159(5):2318-2326). The mutated porcine MHC class I gene is then cloned into pcDNA3 which is modified to contain splice and polyadenylation sites, a neomyocin resistance gene, and a dopamine D2 receptor promoter for expression in the striatum.

Production of Transgenic Pig

Both of the DNA constructs which encode FasL and human NK inhibitory sequence are introduced into the pronuclei of a fertilized oocyte by microinjection as described in detail herein and in Hogan, B. et al., A Laboratory Manual (Cold Spring Harbor Laboratory Press, Cold Spring Harbor, N.Y., 1986). The oocyte is then allowed to develop in a pseudopregnant female foster pig. The foster pig is allowed to carry the fetuses until the desired gestational age.

Upon isolation of the fetuses, the tissues of the transgenic pigs are analyzed for the presence of FasL and NK inhibitory sequence by either directly analyzing RNA or by assaying the tissue. For example, in vitro techniques for detection of FasL or NK inhibitory sequence mRNA include Northern hybridizations and in situ hybridizations. In vitro techniques for detection of FasL protein or a protein encoded by the NK inhibitory sequence include enzyme linked immunosorbent assays (ELISAs), Western blots, immunoprecipitations and immunofluorescence. In vitro techniques for detection of FasL or NK inhibitory sequence genomic DNA include Southern hybridizations. Furthermore, in vivo techniques for detection of FasL protein include introducing into a subject a labeled anti-FasL antibody. For example, the antibody can be labeled with a radioactive marker whose presence and location in a subject can be detected by standard imaging techniques.

Isolation and Transplantation of Striatal Cells Expressing FasL and Killer Inhibitory Sequence

Porcine striatal cells expressing FasL and NK inhibitory sequence are isolated by the methods described in PCT Publication Number WO 96/14399 published on May 17, 1996. Briefly, dissection of the fetal brain is performed in PBS under a dissecting microscope to expose the ganglionic eminences in the basal telencephalon. Tissue fragments derived from both hemispheres of all fetal brains of a litter are pooled. The tissue is incubated in 0.5% trypsin-EDTA in HBSS (Sigma) and DNase at 37° C. for 15 minutes, washed three times with HBSS, then gently triturated through the tips of fire-polished Pasteur pipettes of progressively smaller diameter until a milky suspension is obtained.

The striatal cells are injected into the striatum of patients with Huntington's disease by direct stereotaxic injection. Bjorklund et al. (1983) Acta Physiol. Scand. Suppl. 522:1-75. Assessment of graft survival is monitored by PET imaging and functional recovery is assessed by variations in the patient's symptoms as measured using standard Huntington's disease rating scales.

### Example 7

### Transplantation Of Porcine Cardiomyocytes Expressing Human IL-12 Receptor

Isolation and Modification of Porcine Cardiomyocytes

Porcine cardiomyocytes are isolated using a dissection microscope to expose the heart and gently pulling it free from its attachment to the vasculature. As described in greater detail in PCT Publication Number WO 96/38544 published on Dec. 5, 1996, the hearts are then transferred, using a large bore pipette, to a Petri dish containing a small volume (enough to keep tissue wet) of digestion buffer (0.05% trypsin, 0.05% collagenase P, 0.05% bovine serum albumin (BSA)). The hearts are cut into small pieces with a surgical blade and torn into fine pieces using the needles of two 1 cc syringes. Using a large bore pipette, tissue pieces are then transferred into a 50 ml conical tube and, together with additional volume, are rinsed from the Petri dish, and spun down for 5 minutes at 200×g. Pelleted tissue is then resuspended in 0.4 ml of digestion buffer per heart and is placed at 37° C. water bath with intermittent shaking. After 20 minutes of incubation, the digestion mixture is spun down for 5 minutes at 200×g and is resuspended in the same volume of a fresh digestion buffer and is returned for incubation for another 30 minutes

Myocytes released into the medium after 50 minutes of digestion are transferred into another conical tube and enzyme activity is stopped with equal volume of growth medium: MCDB+dexamethasone, (0.39 μg/ml)+epidermal growth factor (EGF) (10 ng/ml)+15% fetal bovine serum (FBS). Undigested tissue in the digestion tube is washed several times with growth medium and added to the cell harvest. Cells are spun down, resuspended in 2 ml of growth medium for the cell count and then, depending on cell density, seeded into 100 mm tissue culture dishes at approximately 3×10⁵ cells/dish. The growth medium for the cardiomyocytes is MCDB 120+dexamethasone, e.g., 0.39 μg/ml, +Epidermal Growth Factor (EGF), e.g., 10 ng/ml, +fetal calf serum, e.g., 15%.

The cardiomyocytes are genetically modified to express soluble human IL-12 receptor (the nucleotide sequence of which is provided in Chua et al. (1994) J. Immunol. 153:128-136) using a recombinant adenovirus. The genome of an adenovirus is manipulated such that it encodes and expresses IL-12 receptor but is inactivated in terms of its ability to replicate in a normal lytic viral life cycle, as described in greater detail in, for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. The gene encoding IL-12 is linked to chicken β actin promoter and a splice and polyadenylation site and ligated into Ad type 5 dl 324 vector. The cardiomyocytes are infected with the viral vector containing the gene encoding IL-12 receptor by incubating at 37° C. for 24 hours .

Transplantation of Cardiomyocytes Expressing IL-12 Receptor

The cardiomyocytes expressing human IL-12 receptor are administered to a recipient by direct injection of the cardiomyocytes into the ventricular myocardium. The recipient is a mammal, e.g., a B6D2/F1 mouse which is recognized by those of skill in the art as an animal model yielding results predictive of results in humans. See, e.g., Soonpaa, M. H. et al. (1994) Science 264:98-101; Koh, G. Y. et al. (1993) Am. J. Physiol. 33:H1727-1733. Cardiomyocyte survival in an allogenic recipient can be measured in vivo by using antibodies to cardio-specific myosin, tropinin or a Y specific probe. In addition, if the porcine cardiomyocytes are transplanted into a xenogeneic recipient, a PRE probe can be used to detect cardiomyocyte survival in vivo.

### Example 8

### Transplantation Of Human Hepatocytes Expressing Human CD40

Isolation of Human Hepatocytes

Hepatocytes are isolated from a donor liver that has not been used for transplantation, e.g., a donor liver which has traumatic damage. The liver is cut into two lobes, the right lobe is processed first while the left lobe is stored on ice and refrigerated until further processing. The liver is then transferred to a tared jar for weighing. The weight is recorded on the Batch Record. The liver is transferred to a biological safety cabinet and placed into a stainless steel pan maintained at 36° C.-40° C. Major vessels are identified for perfusion and perfusion tubing is primed and inserted into the vasculature. All solutions used during this processing of the liver contain a combination of three antibiotics: penicillin, streptomycin and neomycin (50 μg/ml, 50 μg/ml and 100 μg/ml, respectively). The liver is perfused with 2 liters of EDTA solution at 36° C.-40° C. for 15 minutes to 20 minutes at a rate of 75 ml/minute to 100 ml/minute. After 2 liters have been perfused through the liver, the solution is aspirated from the pan, an aliquot provided to Quality Control for bioburden and LAL testing and the remainder discarded. The perfusion tubing is primed with collagenase solution and reinserted into the liver vasculature. One liter of collagenase solution heated to 36° C.-40° C. is perfused at a rate of approximately 100 ml/minute. If the tissue is insufficiently digested at the time the source bottle is depleted, then the solution is recycled and perfused until digestion is complete. The tissue is transferred to a second stainless steel pan for maceration. One liter Ringer's solution at 2° C. to 5° C. is added to the pan and the tissue is macerated manually to release cells from the digested tissue. The digest is filtered through 200 μm polyester sterile mesh into a collection bottle. The digest is further diluted with cold Ringer's solution at a ratio of 10 ml solution for each gram of tissue processed. The hepatocytes are washed three times by centrifuging at 40 G for 4 minutes at 5° C. After each centrifugation, the supernant is aspirated and the cells resuspended in fresh Ringer's stop medium to formulate a dose of 200 ml containing 2×10⁹ cells. The cells are suspended in University of Wisconsin (UW) medium and are infected with the viral vector containing the gene encoding CD40L by incubating at 37° C. for 24 hours. The hepatocytes are then resuspended in fresh UW medium.

Modification of Human Hepatocytes

Hepatocytes are genetically modified to express CD40 using a recombinant adenovirus. The genome of an adenovirus is manipulated such that it encodes and expresses CD40 but is inactivated in terms of its ability to replicate in a normal lytic viral life cycle, described in greater detail in, for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g. Ad2, Ad3, Ad7 etc.) are well known to those skilled in the art. The human CD40 gene (the nucleotide sequence which is provided in Stamenlovic et al. (1988) EMBO J. 7:1053-1059) is fused to the constant domain and secretory signal of Ig by methods known in the art. The CD40/Ig fusion product having BamH1/Xhol restriction sites at the 5′ and 3′ ends is spliced into the pcDNA3 expression vector (Invitrogen, San Diego, Calif.) which is modified to contain an albumin promoter.

Transplantation of Human Hepatocytes Expressing CD40 into a Human Recipient

The isolated hepatocytes expressing CD40 are transplanted into an infant born with a urea cycle enzyme deficiency which causes hyperammonemia. Briefly, the human recipient is placed under general anesthesia and an umbilical vein catheter is placed. Pressure monitoring is established for portal vein pressures and the liver is perfused with heparinized saline solution at 5 cc/hour. Non-invasive monitoring of the patient's oxygen saturation and an EKG are maintained throughout the procedure. Infusion of the hepatocytes is done by hand to allow for continuous rocking of the syringe to keep the hepatocytes in suspension. 2×10⁹ hepatocytes are suspended in saline solution and administered at approximately 15 cc every 5 minutes. Every 5 minutes, portal blood pressure is measured. After completion of the hepatocyte infusion, the umbilical catheter remains in place for 24 hours. Immunosuppressive drugs, including cyclosporine, azathioprine and prednisone, are administered the same as are routinely administered for an orthotopic liver transplant. In addition, other antibiotics and antiviral agents are administered into the umbilical catheter following Transplant Unit Protocols. Graft survival is assessed by measuring serum ammonium levels in the patient.

Hepatocytes expressing CD40 can also be transplanted into an adult human recipient by the methods described in Strom et al. (1997) Transplantation 63(4):559-569.

### Example 9

### Transplantation Of Porcine Hepatocytes Expressing A Fusion Protein Comprising Fas Ligand And A Modified Porcine MHC Class I Killer Inhibitory Sequence

A gene encoding a fusion protein is produced such that the first portion contains cDNA encoding human FasL and the second portion is a porcine MHC class I gene modified as described in Example IV. In addition, the cDNA sequence encoding human FasL is described in Takahashi et al. (1994) Cell 76:969-976. The fusion gene is linked to an albumin promoter for liver specific expression and cloned into a pcDNA3 vector (Invitrogen, San Diego, Calif.) which is modified to contain a polyadenylation site.

Production of Transgenic Pig

The purified DNA construct encoding the fusion protein is introduced into the pronuclei of a fertilized oocyte by microinjection, as described in detail herein and in Hogan, B. et al., A Laboratory Manual (Cold Spring Harbor Laboratory Press, Cold Spring Harbor, N.Y., 1986). The oocyte is allowed to develop in a pseudopregnant female foster pig. The foster pig is allowed to carry the fetuses to term.

Upon birth of the litter, the tissues of the transgenic pigs are analyzed for the presence of the fusion protein by either directly analyzing RNA, assaying the tissue for FasL or NK inhibitory sequence, or by assaying conditioned medium for secreted FasL/NK inhibitory sequence protein. For example, in vitro techniques for detection of FasL or NK inhibitory sequence mRNA include Northern hybridizations and in situ hybridizations. In vitro techniques for detection of FasL protein or a protein encoded by the NK inhibitory sequence include enzyme linked immunosorbent assays (ELISAs), Western blots, immunoprecipitations and immunofluorescence. In vitro techniques for detection of FasL or NK inhibitory sequence genomic DNA include Southern hybridizations. Furthermore, in vivo techniques for detection of FasL protein include introducing into a subject a labeled anti-FasL antibody. For example, the antibody can be labeled with a radioactive marker whose presence and location in a subject can be detected by standard imaging techniques.

Isolation and Transplantation of Hepatocytes Expressing FasL/Killer Inhibitorv Fusion Protein

Porcine hepatocytes are isolated by the two stage perfusion technique originally described by Berry and Friend ((1969) J. Cell Biol. 43:506-520) and modified by others (Maganto P. et al. (1992) Transplant Proc. 24:2826-2827; Gerlach J. C. et al. (1994) Transplantation 57:1318-1322) for ex vivo perfusion of large animal organs and described in detail in WO 96/37602 published on Nov. 28, 1996. A liver lobe of 100-200 g is cannulated and perfused with HBSS (minus Mg⁺⁺, Ca⁺⁺) containing 0.4 mM EDTA, 10 mM HEPES, pH 7.4 and penicillin (100 U/ml)-streptomycin (100 ug/ml) at 35° C. This is followed by a second perfusion with complete HBSS containing collagenase P (0.8 mg/ml, Boehringer Mannheim), 10 mM HEPES, pH 7.4, and penicillin-streptomycin at 35° C. The perfusion is continued until visible softening of the organ occurs. The total time for digestion ranges from 12-20 minutes. The digested liver is then physically disrupted and the released hepatocytes are washed (50×g) twice in DMEM/Weymouth media containing 10% heat inactivated calf serum at 4° C.

Porcine hepatocytes are collected and counted. Viability is assessed by trypan blue staining. The purity of the hepatocyte preparation is judged by immunofluorescence for class II bearing non-parenchymal cells. Purity determinations are made by counting the positive staining cells (monoclonal antibody ISCR3) in several fields consisting of 200 cells.

The isolated porcine hepatocytes expressing FasL/Killer inhibitory sequence fusion protein are transplanted into a patient having chronic end-stage liver disease with acute decompensation or acute liver failure with pathologic verified diagnosis. Strom et al. (1997) Transplantation 63(4):559-569. Specifically, the cells are infused into the splenic artery of the recipient and graft survival is assessed by measuring serum anmmonia levels in the recipient as described in Strom et al., supra.

In addition, the hepatocytes expressing FasL/Killer inhibitory sequence fusion protein can be transplanted into an infant born with urea cycle enzyme deficiency which causes hyperammonemia. Briefly, the recipient is placed under general anesthesia and an umbilical vein catheter is placed. Pressure monitoring is established for portal vein pressures and the liver is perfused with heparinized saline solution at 5 cc/hour. Non-invasive monitoring of the patient's oxygen saturation and an EKG are maintained throughout the procedure. Infusion of the hepatocytes is done by hand to allow for continuous rocking of the syringe to keep the hepatocytes in suspension. 2×10⁹ hepatocytes are suspended in saline solution and administered at approximately 15 cc every 5 minutes. Every 5 minutes, portal blood pressure is measured. After completion of the hepatocyte infusion, the umbilical catheter remains in place for 24 hours. Antibiotics and antiviral agents are administered into the umbilical catheter following Transplant Unit Protocols. Graft survival is assessed by measuring serum ammonium levels in the patient.

### Example 10

### Methods Of Producing Essentially Pathogen-Free Swine From Which Cells Of The Invention Can Be Obtained

A. Collecting, Processing, and Analyzing Pig Fecal Samples for Signs of Pathogens

Feces are extracted from the pig's rectum manually and placed in a sterile container. About a 1.5 cm diameter portion of the specimen was mixed thoroughly in 10 ml of 0.85% saline. The mixture is then strained slowly through a wire mesh strainer into a 15 ml conical centrifuge tube and centrifuged at 650×g for 2 minutes to sediment the remaining fecal material. The supernatant is decanted carefully so as not to dislodge the sediment. and 10% buffered formalin was added to the 9 ml mark, followed by thorough mixing. The mixture is allowed to stand for 5 minutes. 4 ml of ethyl acetate is added to the mixture and the mixture is capped and mixed vigorously in an inverted position for 30 seconds. The cap is then removed to allow for ventilation and then replaced. The mixture is centrifuged at 500×g for 1 minute (four layers should result: ethyl acetate, debris plug, formalin and sediment). The debris plug is rimmed using an applicator stick. The top three layers are carefully discarded by pouring them off into a solvent container. The debris attached to the sides of the tube is removed using a cotton applicator swab. The sediment is mixed in either a drop of formalin or the small amount of formalin which remains in the tube after decanting. Two separate drops are placed on a slide to which a drop of Lugol's iodine is added. Both drops are coverslipped and carefully examined for signs of pathogens, e.g., protozoan cysts of trophozoites, helminth eggs and larvae. Protozoan cyst identification is confirmed, when required, by trichrome staining.

B. Co-cultivation Assay for Detecting the Presence of Human and Animal Viruses in Pig Cells

Materials

Cell Lines

African green monkey kidney, (VERO), cell line American Type Culture Collection, (ATCC CCL81), human embryonic lung fibroblasts, (MRC-5) cell line American Type Culture Collection, (ATCC CCL 171), porcine kidney, (PK-15), cell line American Type Culture Collection, (ATCC CRL 33), porcine fetal testis, (ST), cell line American Type Culture Collection, (ATCC CRL 1746).

Medium, Antibiotics, and Other Cells, and Equipment

Fetal calf serum, DMEM, Penicillin 10,000 units/ml, Streptomycin 10 mg/ml, Gentamicin 50 mg/ml, guinea pig erythrocytes, chicken erythrocytes, porcine erythrocytes,

Negative Control (sterile cell culture medium), Positive Controls: VERO and MRC-5 Cells:

Poliovirus type 1 attenuated, (ATCC VR-1 92) and Measles virus, Edmonston strain, (ATCC VR-24), PK-1 5 and ST Cells: Swine influenza type A, (ATCC VR-99), Porcine Parvovirus, (ATCC VR-742), and Transmissible gastroenteritis of swine, (ATCC VR-743). Equipment: tissue Culture Incubator, Inverted Microscope, Biological Safety Cabinet.

These materials can be used in a co-cultivation assay (a process whereby a test article is inoculated into cell lines (VERO, MRC-5, PK1 5, and ST) capable of detecting a broad range of human, porcine and other animal viruses). Hsuing, G. D., “Points to Consider in the Characterization of Cell Lines Used to Produce Biologicals” in Diagnostic Virology, 1982 (Yale University Press, New Haven, Conn., 1982).

Experimental Design and Methodology

A total of three flasks (T25) of each cell line are inoculated with at least 1 ml of test article. Three flasks of each cell line can also be inoculated with the appropriate sterile cell culture medium as a negative control. Positive control viruses are inoculated into three flasks of each cell line. After an absorption period, the inoculate is removed and all flasks incubated at 35-37° C. for 21 days. All flasks are observed at least three 35 times per week for the development of cytopathic effects, (CPE), of viral origin. Harvests are made from any flasks inoculated with the test article that show viral CPE.

At Day 7 an aliquot of supernatant and cells from the flasks of each test article are collected and at least 1 ml is inoculated into each of three new flasks of each cell line. These subcultures are incubated at 35-37° C. for at least 14 days. All flasks are observed and tested as described above.

At Day 7, the flasks from each test article are also tested for viral hemadsorption. (HAd), using guinea pig, monkey and chicken erythrocytes at 2-8° C. and 35-37° C. at 14 days postinoculation.

At Day 21, if no CPE is noted, an aliquot of supernatant from each flask is collected, pooled, and tested for viral hemagglutination, (HA), using guinea pig, monkey, and chicken erythrocytes at 2-8° C. and 35-37° C. Viral identification is based on characteristic viral cytopathic effects (CPE) and reactivity in HA testing.

The test samples are observed for viral cytopathic effects in the following manner: All cultures are observed for viral CPE at least three times each week for a minimum of 21 days incubation. Cultures are removed from the incubator and observed using an inverted microscope using at least 40×magnification. 100× or 200×magnification is used as appropriate. If any abnormalities in the cell monolayers, including viral CPE, are noted or any test articles cause total destruction of the cell monolayer, supernatant and cells are collected from the flasks and samples are subcultured in additional flasks of the same cell line. Samples can be stored at −60° to −80° C. until subcultured. After 7 and 14 days incubation, two blind passages are made of each test article by collecting supernatant and cells from all flasks inoculated with each sample. Samples can be stored at −60° to −80° C. until subcultured.

Hemadsorbing viruses are detected by the following procedure: after 21 days of incubation, a hemadsorption test is performed on the cells to detect the presence of hemadsorbing viruses. The cells are washed 1-2 times with approximately 5 mls of PBS. One to two mls of the appropriate erythrocyte suspension (either guinea pig, porcine, or chicken erythrocytes), prepared as described below, is then added to each flask. The flasks are then incubated at 2-8° C. for 15-20 minutes, after which time the unabsorbed erythrocytes are removed by shaking the flasks. The erythrocytes are observed by placing the flasks on the lowered stage of a lab microscope and viewing them under low power magnification. A negative result is indicated by a lack of erythrocytes adhering to the cell monolayer. A positive result is indicated by the adsorption of the erythrocytes to the cell monolayer.

Hemagglutination testing, described in detail below, is also performed after 21 days of incubation of the subcultures. Viral isolates are identified based on the cell line where growth was noted, the characteristics of the viral CPE, the hemadsorption reaction, and hemagglutination reactions, as appropriate. The test article is considered negative for the presence of a viral agent, if any of the cell lines used in the study demonstrate viral, CPE, HA, or HAd in a valid assay.

C. Procedure for Preparing and Maintaining Cell Lines Used to Detect Viruses in Pig Cells

Materials

Fetal calf serum (FCS), DMEM, Penicillin 10,000 unit/ml, Streptomycin 10 mg/ml, Gentamicin 50 mg/ml, T25 tissue culture flasks, tissue culture incubator (5% C0₂, 37° C.)

Procedure

Aseptic techniques are followed when performing inoculations and transfers. All inoculations and transfers are performed in a biological safety cabinet. Media is prepared by adding 10% FCS for initial seeding, 5% FCS for maintenance of cultures, as well as 5.0 ml of penicillin/streptomycin and 0.5 ml of gentamicin per 500 ml media. Sufficient media is added to cover the bottom of a T25 tissue culture flask. The flask is seeded with the desired cell line and incubated at 37° C., 5% CO₂ until cells are 80 to 100% confluent. The flasks are then inoculated with virus (QCP25).

D. Preparation of Erythrocyte (rbc) Suspensions Used in Hemadsorption (HAd) and Hemagglutination (HA) Virus Detection Testing

Materials

Phosphate buffered saline, (PBS), pH 7.2, guinea pig erythrocytes stock solution, porcine erythrocytes stock solution, chicken erythrocytes stock solution, sterile, disposable centrifuge tubes, 15 or 50 ml Laboratory centrifuge

Procedure

An appropriate amount of erythrocytes (rbc) is obtained from stock solution. The erythrocytes are washed 3 times with PBS by centriftigation at approximately 1000×g for 10 minutes. A 10% suspension is prepared by adding 9 parts of PBS to each one part of packed erythrocytes. The 10% rcb suspensions are stored at 2-8° C. for no more than one week. 0.5% ecb suspensions are prepared by adding 19 parts of PBS to each one part of 10% rbc suspension. Fresh 0.5% rbc suspensions are prepared prior to each day's testing.

### Hemagglutination (HA) Test

A hemagglutination test is a test that detects viruses with the property to agglutinate erythrocytes, such as swine influenza type A, parainfluenza, and encephalomyocarditis viruses, in the test article. Hsuing, G. D. (1982) Diagnostic Virology (Yale University Press, New Haven, Conn.);. Stites, Daniel P and Terr, Abba I, (1991), Basic and Clinical Immunology (Appleton & Lange, East Norwalk, Conn.).

Materials

Supernatants from flasks of the VERO cell line, MRC-5 inoculated with the test article, flasks of positive and negative controls, phosphate buffered saline (PBS), pH 7.2, guinea pig erythrocytes (GPRBC), 0.5% suspension in PBS, chicken erythrocytes (CRBC), 0.5% suspension in PBS, porcine erythrocytes (MRBC), 0.5% suspension in PBS

Procedure

All sample collection and testing is performed in an approved biological safety cabinet. 0.5% suspensions of each type of erythrocytes are prepared as described above. The HA test on all cell lines inoculated with samples of the test articles at least 14 days post-inoculation. Positive and negative control cultures are included for each sample and monolayers are examined to ensure that they are intact prior to collecting samples.

At least 1 ml of culture fluid from each flask inoculated with the test article is collected and pooled. 1 ml samples from the negative and positive control cultures are also collected and pooled. A set of tubes is labeled with the sample number and type of erythrocyte (distinguish positive and negative suspension) to be added. Racks may be labeled to differentiate the type of erythrocyte. 0.1 ml of sample is added to each tube. 0.1 ml of the appropriate erythrocyte suspension is added to each tube. Each tube is covered with parafilm and mixed thoroughly. One set of tubes is incubated at 2-8° C. until tight buttons form in the negative control in about 30-60 minutes. Another set of tubes is incubated at 35-37° C. until tight buttons form in the negative control in about 30-60 minutes.

Formation of a tight button of erythrocytes indicates a negative result. A coating of the bottom of the tube with the erythrocytes indicates a positive result.

E. Methods Used for Fluorescent Antibody Stain of Cell Suspensions Obtained from Flasks Used in Detection of Viruses in Porcine Cells Using Cell Culture Techniques (as Described in Sections B and C)

Materials

Pseudorabies, parvovirus, enterovirus, adenovirus, transmissible Gastroenteritis Virus. bovine viral diarrhea, encephalomyocarditis virus, parainfluenza, vesicular stomatitis virus., microscope slides, PBS, incubator with humidifying chamber at 36° C., Evan's blue coutner stain, DI Water, fluorescent microscope, trypsin, serum containing media, acetone, T25 Flask.

Procedure

Cells (described in Sections B and C) are trypsinized to detach them from the T25 flask and sufficient media is added to neutralize trypsin activity. A drop of cell suspension is placed on each microscope slide and allowed to air dry. A slide for each fluorescent antibody is prepared. Cells are fixed by immersion in acetone for five minutes. Each fluorescent antibody solution is placed on each slide to cover cells and the slides are incubated in humidifying chamber in incubator at 36° C. for 30 minutes. The slides are then washed in PBS for five minutes. The wash is repeated in fresh PBS for five minutes followed by a rinse with DI water.

The cells are counterstained by placing Evan's blue solution on each slide to cover cells for five minutes at room temperature. The slides are then washed in PBS for five minutes. The wash is repeated in fresh PBS for five minutes followed by a rinse with DI water. The slides are then allowed to air dry. Each slide is inspected under a fluorescent microscope. Any fluorescent inclusion bodies characteristic of infection are considered a positive result for the presence of virus.

F. Proceduresfor Defining Bacteremic Pigs

Materials

Anaerobic BMB agar (5% sheep blood, vitamin K and hemin [BMB/blood]), chocolate Agar with Iso Vitalex, Sabaroud dextrose agar/Emmons, 70% isopropyl alcohol swabs, betadine solution, 5% CO₂ incubator at 35-37° C., anaerobic blood agar plate, gram stain reagents (Columbia Broth Media), aerobic blood culture media (anaerobic brain heart infusion with vitamin K& hemin), septicheck media system, vitek bacterial identification system, laminar flow hood, microscope, and bacteroids and Bacillus stocks

Procedure

Under a laminar flow hood, disinfect the tops of bottles for aerobic and anaerobic blood cultures of blood obtained from pig with 70% isopropyl alcohol, then with betadine The rubber stopper and cap from the aerobic blood culture bottle are removed and a renal septicheck media system is attached to the bottle. The bottles are incubated in 5% C0₂ for 21 days at 35-37° C., and observed daily for any signs of bacterial growth (i.e. gas bubbles, turbidity, discoloration or discrete clumps). Negative controls consisting of 5 cc of sterile saline in each bottle and positive controls consisting of Bacillus subtilis in the aerobic bottle and Bacteriodes Vulgaris in the anaerobic bottle are used. If signs of bacterial growth are observed, a Gram stain is prepared and viewed microscopically at 100×oil immersion for the presence of any bacteria or fungi. The positive bottles are then subcultured onto both chocolate agar plates with Iso Vitlex and onto BMB plates. The chocolate plate is incubated at 35-37° C. in 5% CO₂ for 24 hours and the BMB anaerobically at 35-37° C. for 48 hours. Any yeast or fungi that is in evidence at gram stain is subcultured onto a Sabaroud dextrose/Emmons plate. The Vitek automated system is used to identify bacteria and yeast. Fungi are identified via their macroscopic and microscopic characteristic. If no signs of growth are observed at the end of 21 days, gram stain is prepared and observed microscopically for the presence of bacteria and fungi.

Absence of growth in the negative control bottles and presence of growth in the positive control bottles indicates a valid test. The absence of any signs of growth in both the aerobic and anaerobic blood culture bottles, as well as no organisms seen on gram stain indicates a negative blood culture. The presence and identification of microorganism(s) in either the aerobic or anaerobic blood culture bottle indicates of a positive blood culture; this typically is due to a bacteremic state.

Equivalents

Those skilled in the art will recognize. or be able to ascertain using no more than routine experimentation, many equivalents of the specific embodiments of the invention described herein. Such equivalents are intended to be encompassed by the following claims.

What is claimed is:

1. A transplantable composition comprising a cell which is genetically modified to express an immunoregulatory molecule which inhibits T cell activation selected from the group consisting of: CD8, soluble cytokine receptor, soluble costimulatory molecule, soluble CD40 and soluble CD40L and/or a molecule comprising a killer inhibitory sequence selected from the group consisting of: a human MHC class I molecule, a chimeric MHC class I molecule, and a viral MHC class I homolog, such that following transplantation of the cell into a human subject, rejection of the cell is inhibited.

2. A transplantable composition comprising a cell which is genetically modified to express a first immunoregulatory molecule which inhibits T cell activation and a second immunoregulatory molecule which comprises a killer inhibitory sequence, such that following transplantation of the cell into a human subject, rejection of the cell is inhibited.

3. A transplantable composition comprising a xenogeneic cell which is genetically modified to express an immunoregulatory molecule which inhibits T cell activation selected from the group consisting of CD8, a soluble cytokine receptor, a soluble costimulatory molecule, soluble CD40 and soluble CD40L, such that following transplantation of the cell into a human subject, rejection of the xenogeneic cell is inhibited.

4. The composition of claim 3, wherein the first and second immunoregulatory molecules are expressed as a single soluble fusion protein.

5. The composition of claim 3, wherein the first or second immunoregulatory molecule is expressed on the surface of the cell.

6. The composition of claim 3, wherein the first immunoregulatory molecule is secreted by the cell.

7. The composition of claim 3, wherein the cell is genetically modified by transfection of one or more heterologous nucleic acid molecules encoding the first and second immunoregulatory molecules such that the first and second molecules are expressed by the cell.

8. The composition of claim 3, wherein the first immunoregulatory molecule is selected from the group consisting of FasL, CD8, a soluble cytokine receptor, a soluble costimulatory molecule, soluble CD40 and soluble CD40L.

9. The composition of claim 3, wherein the second immunoregulatory molecule is selected from the group consisting of a human MHC class I molecule, a chimeric MHC class I molecule, and a viral MHC class I homolog.

10. The composition of claim 3, wherein the expression of the first or second immunoregulatory molecule is under the control of a tissue specific promoter.

11. The composition of claim 3, wherein the cell is selected from the group consisting of: a fetal cell, a stem cell, an embryonic stem cell, and a progenitor cell.

12. The composition of claim 3, wherein the cell is obtained from a pig which is predetermined to be free from at least one organism selected from the group consisting of zoonotic and cross-placental organisms.

13. The composition of claim 3, wherein the cell is selected from the group consisting of: a pancreatic islet cell, a kidney cell, a cardiac cell, a muscle cell, a liver cell, a lung cell, an endothelial cell, a central nervous system cell, a peripheral nervous system cell, an epithelial cell, an eye cell, a skin cell, an ear cell, and a hair follicle cell.

14. A transplantable composition comprising a cell which is genetically modified to express an immunoregulatory molecule selected from the group consisting of: a chimeric MHC class I molecule and a viral MHC class I homolog, such that following transplantation of the cell into a subject, rejection of the cell is inhibited.

15. The composition of claim 14, wherein the expression of the immunoregulatory molecule is under the control of a tissue specific promoter.

16. The composition of claim 14, wherein the cell is selected from the group consisting of: a fetal cell, a stem cell, an embryonic stem cell, and a progenitor cell.

17. The composition of claim 14, wherein the cell is obtained from a pig which is predetermined to be free from at least one organism selected from the group consisting of zoonotic and cross-placental organisms.

18. The composition of claim 14, wherein the cell is selected from the group consisting of: a pancreatic islet cell, a kidney cell, a cardiac cell, a muscle cell, a liver cell, a lung cell, an endothelial cell, a central nervous system cell, a peripheral nervous system cell, an epithelial cell, an eye cell, a skin cell, an ear cell, and a hair follicle cell.

19. The composition of claim 3 further comprising a pharmaceutically acceptable carrier.

20. A method for inhibiting immune rejection of a cell comprising administering a cell which has been genetically modified to express an immunoregulatory molecule which inhibits T cell activation selected from the group consisting of CD8, soluble cytokine receptor, soluble costimulatory molecule, soluble CD40 and soluble CD40L, such that following transplantation of the cell into a subject, rejection of the cell is inhibited.

21. A method for inhibiting immune rejection of a cell comprising administering a cell which is genetically modified to express an immunoregulatory molecule which inhibits T cell activation selected from the group consisting of: CD8, soluble cytokine receptor, soluble costimulatory molecule, soluble CD40 and soluble CD40L and/or a molecule comprising a killer inhibitory sequence selected from the group consisting of: a human MHC class I molecule, a chimeric MHC class I molecule, or a viral MHC class I homolog, such that following transplantation of the cell into a human subject, rejection of the cell is inhibited.

22. A method for inhibiting immune rejection of a cell comprising administering a cell which has been genetically modified to express an immunoregulatory molecule which inhibits T cell activation selected from the group consisting of CD8, soluble cytokine receptor, soluble costimulatory molecule, soluble CD40 and soluble CD40L, such that following transplantation of the cell into a subject, rejection of the cell is inhibited.

23. A method for inhibiting immune rejection of a cell comprising administering a cell which has been genetically modified to express a first immunoregulatory molecule which inhibits T cell activation and a second immunoregulatory molecule which comprises a killer inhibitory sequence, such that following transplantation of the cell into a human subject, immune rejection of the cell is inhibited.

24. The method of claim 23, wherein the first and second immunoregulatory molecules are expressed as a single soluble fusion protein.

25. The method of claim 23, wherein the first or second immunoregulatory molecule is expressed on the surface of the cell.

26. The method of claim 23, wherein the first immunoregulatory molecule is secreted by the cell.

27. The method of claim 23, wherein the cell is genetically modified by transfection of one or more heterologous nucleic acid molecules encoding the first and second immunoregulatory molecules such that the first and second molecules are expressed by the cell.

28. The method of claim 23, wherein the first immunoregulatory molecule is selected from the group consisting of FasL, CD8, a soluble cytokine receptor, a soluble costimulatory molecule, soluble CD40 and soluble CD40L.

29. The method of claim 23, wherein the second immunoregulatory molecule is selected from the group consisting of a human MHC class I molecule, a chimeric MHC class I molecule, and a viral MHC class I homolog.

30. The method of claim 23, wherein the expression of the first or second immunoregulatory molecule is under the control of a tissue specific promoter.

31. The method of claim 23, wherein the cell is selected from the group consisting of: a fetal cell, a stem cell, an embryonic stem cell, and a progenitor cell.

32. The method of claim 23, wherein the cell is obtained from a pig which is predetermined to be free from at least one organism selected from the group consisting of zoonotic and cross-placental organisms.

33. The method of claim 23, wherein the cell is selected from the group consisting of: a pancreatic islet cell, a kidney cell, a cardiac cell, a muscle cell, a liver cell, a lung cell, an endothelial cell, a central nervous system cell, a peripheral nervous system cell, an epithelial cell, an eye cell, a skin cell, an ear cell, and a hair follicle cell.

34. A method for inhibiting immune rejection of a cell comprising administering a cell which has been genetically modified to express a chimeric MHC class I molecule and a viral MHC class I homolog, such that following transplantation of the cell into a human subject, immune rejection of the cell is inhibited.

35. The method of claim 34, wherein the expression of the immunoregulatory molecule is under the control of a tissue specific promoter.

36. The method of claim 34, wherein the cell is selected from the group consisting of: a fetal cell, a stem cell, an embryonic stem cell, and a progenitor cell.

37. The method of claim 34, wherein the cell is obtained from a pig which is predetermined to be free from at least one organism selected from the group consisting of zoonotic and cross-placental organisms.

38. The method of claim 34, wherein the cell is selected from the group consisting of: a pancreatic islet cell, a kidney cell, a cardiac cell, a muscle cell, a liver cell, a lung cell, an endothelial cell, a central nervous system cell, a peripheral nervous system cell, an epithelial cell, an eye cell, a skin cell, an ear cell, and a hair follicle cell.

39. The method of claim 34, further comprising the step of administering to the subject an immunoregulatory molecule which is capable of inhibiting T cell or natural killer cell mediated immune rejection of the cell.

40. A non-human transgenic animal comprising a cell which is genetically modified to express a chimeric MHC class I molecule or a viral MHC class I homolog, such that following transplantation of the cell into a human subject, immune rejection of the cell is inhibited.

41. A non-human transgenic animal comprising a cell which is genetically modified to express a first immunoregulatory molecule which inhibits T cell activation and a second immunoregulatory molecule which comprises a killer inhibitory sequence, such that following transplantation of the cell into a human subject, immune rejection of the cell is inhibited.
